(19) United States
(12) Patent Application Publication
(10) Pub. No.: US 2026/0139052 A1
(43) Pub. Date: May 21, 2026

(54) ANTI-CTLA-4 ANTIBODY DOSING REGIMENS

(71) Applicant: OncoC4, Inc., Rockville, MD (US)

(72) Inventors: Yang Liu, Potomac, MD (US); Pan Zheng, Potomac, MD (US); Martin Devenport, Gaithersburg, MD (US)

(21) Appl. No.: 18/705,924

(22) PCT Filed: Oct. 28, 2022
(86) PCT No.: PCT/US2022/078875

§ 371 Date: Apr. 29, 2024

Related U.S. Application Data
(60) Provisional application No. 63/273,660, filed on Oct. 29, 2021.

(51) Int. Cl. C07K 16/28; A61K 9/00; A61K 39/00; A61P 35/00

(52) U.S. Cl. CPC C07K 16/2818; A61K 9/0019; A61P 35/00; A61K 2039/505; A61K 2039/54; A61K 2039/545; C07K 2317/565

(57) ABSTRACT

The present invention relates to anti-CTLA-4 antibody dosing regimens, including their use in treating cancer.

### FEDERALLY SPONSORED RESEARCH OR DEVELOPMENT

This invention was made in part with Government support under Grant Number R44CA250824 awarded by National Cancer Institute, NIH. The Government has certain rights in this invention.

### FIELD OF THE INVENTION

The invention relates to dosing regimens for anti-CTLA-4 antibodies, including for treating cancer.

### BACKGROUND OF THE INVENTION

Cytotoxic T-lymphocyte-associated protein 4 (CTLA-4), also known as CD152 (cluster of differentiation 152), is a cell surface protein receptor that interacts with B7-1 (CD80) and B7-2 (CD86) to ensure proper function of regulatory T cells and protects host against autoinflammatory diseases. Anti-CTLA-4 monoclonal antibodies (mAbs) such as the approved antibody, ipilimumab (marketed as YERVOY® by Bristol Myers Squibb), have demonstrated strong and broad cancer immunotherapeutic effects (CITE) in a variety of preclinical models and are used clinically both as monotherapy and as part of combination therapy with nivolumab (anti-PD-1, marketed as OPDIVO® by Bristol Myers Squibb). However, CTLA-4 monotherapy has more immunotherapy-related adverse effects (irAEs) than anti-PD-1/PD-L1 therapy. In addition, the rate of severe irAE (Grades 3 and 4) reached 55% in melanoma patients receiving combination of ipilimumab and nivolumab. The strong irAEs further limit the number of doses tolerated by cancer patients. Nevertheless, combination with anti-PD-1 resulted in significantly improved response rates and patient survival in multiple types of cancer. Furthermore, anti-CTLA-4 antibodies induce long-lasting immunity in cancer patients. Therefore, CTLA-4 remains an important immunotherapy target, but major challenges remain in improving both safety and efficacy of anti-CTLA-4 mAbs.

ONC-392 is a highly selective, humanized monoclonal IgG1-kappa isotype antibody against CTLA-4. Recently, it was demonstrated that ONC-392 dissociates from CTLA-4 under low pH to allow its escape from lysosomal degradation and recycling to the cell surface. There are several lines of evidence supporting the notion that a pH-sensitive antibody like ONC-392 is not only safer, but also more effective in Treg depletion and tumor rejection than ipilimumab, which is pH-insensitive. First, by preserving CTLA-4 on the cell surface, ONC-392 leaves a higher ligand density for better ADCC. Second, ONC-392 is more efficient in Treg depletion in the tumor microenvironment. And third, ONC-392 is significantly more potent in inducing rejection of large tumors. Yet, there is a need in the art for an appropriate dosing schedule to provide safe and effective treatments using ONC-392.

### SUMMARY OF THE INVENTION

Provided herein is a method of administering an anti-CTLA-4 antibody, which may comprise administering one or more doses of the anti-CTLA-4 antibody to a subject. The subject may have a cancer. Also provided herein is a method of treating a cancer in a subject in need thereof, which may comprise administering one or more doses of an anti-CTLA-4 antibody to the subject. Further provided are an anti-CTLA-4 antibody for use in treating a cancer, a composition comprising an anti-CTLA-4 antibody for treating a cancer, and use of an anti-CTLA-4 antibody in the manufacture of a medicament for treating a cancer. The anti-CTLA-4 antibody may be used in combination with a second anti-cancer agent, which may be pembrolizumab.

Each dose of the anti-CTLA-4 antibody administered to the subject may independently be about 0.1 mg/kg, 0.3 mg/kg, 1 mg/kg, 3 mg/kg, 6 mg/kg, 10 mg/kg, 15 mg/kg, or 20 mg/kg. The anti-CTLA-4 antibody may be administered once about every 1, 2, 3, 4, 5, or 6 weeks, particularly once about every 3 weeks. Each dose of the anti-CTLA-4 antibody may be about 3 mg/kg, 6 mg/kg, or 10 mg/kg. A first dose of about 10 mg/kg, a second dose of about 10 mg/kg, and one or more subsequent doses of about 1-6 mg/kg may be administered to the subject. Each subsequent dose may be about 6 mg/kg or 3 mg/kg.

The anti-CTLA-4 antibody may be administered to maintain a peak concentration (Cₘₐₓ) of the antibody of about 200-300 μg/mL, particularly about 225-250 μg/mL, and more particularly 225 μg/mL or 250 μg/mL. A dose of the anti-CTLA-4 antibody administered to the subject may be reduced as compared to an immediately preceding dose if (a) the Cₘₐₓ of the anti-CTLA-4 antibody in a blood sample from the subject exceeds 225, 250 or 300 μg/mL; (b) the subject experiences a limiting toxicity; or, (c) the subject is being treated for a cancer and achieves partial or complete response to treatment with the anti-CTLA-4 antibody according to Response Evaluation Criteria in Solid Tumors (RECIST) 1.1 criteria. The anti-CTLA-4 antibody may be administered intravenously, or the composition or medicament may be intended for intravenous administration.

The anti-CTLA-4 antibody may comprise (a) a light chain variable region comprising a complementarity determining region (CDR) 1 comprising the amino acid sequence set forth in SEQ ID NO: 1; a CDR2 comprising the amino acid sequence set forth in any one of SEQ ID NOs: 2-4; and, a CDR3 comprising the amino acid sequence set forth in SEQ ID NO: 5; and, (b) a heavy chain variable region comprising comprising a CDR1 comprising the amino acid sequence set forth in SEQ ID NO: 6; a CDR2 comprising the amino acid sequence set forth in any one of SEQ ID NOs: 7-9; and, a CDR3 comprising the amino acid sequence set forth in SEQ ID NO: 10. The anti-CTLA-4 antibody may comprise a light chain variable region comprising a CDR2 comprising the sequence set forth in SEQ ID NO: 3 and heavy chain variable region comprising a CDR2 comprising the sequence set forth in SEQ ID NO: 9. The anti-CTLA-4 antibody may comprise a light chain variable region comprising the sequence set forth in SEQ ID NO: 12 and a heavy chain variable region comprising the sequence set forth in SEQ ID NO: 16. The anti-CTLA-4 antibody may comprise a light chain comprising the sequence set forth in SEQ ID NO: 23, and a heavy chain comprising the sequence set forth in SEQ ID NO: 21. The anti-CTLA-4 antibody may be ONC-392.

The cancer may be a solid tumor. The cancer may be advanced or metastatic. The subject may have previously exhibited failure or intolerance to standard of care for the cancer. The cancer may be refractory or resistant to anti-PD-1/PD-L1 treatment. The cancer may be melanoma, metastatic melanoma, PD (L)-1-refractory melanoma, non-small cell lung adenocarcinoma, metastatic NSCLC, NSCLC with driver mutations (for example, EGFR/ALK mutations or other targetable mutations), PD-1-refractory NSCLC, head and neck cancer, adenoid cystic carcinoma (which may be R/M), squamous carcinoma, triple negative (basal-type) breast cancer, pancreatic cancer, renal cell carcinoma, cervical cancer, endometrial cancer, colon cancer, hepatocellular carcinoma, other solid tumors, or metastatic colorectal cancer (which may have microsatellite instability).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a diagram of Part A Phase IA of the ONC-392 monotherapy clinical trial. DLT=dose-limiting toxicity; Gr=Grade; Q3W=every 3 weeks; RECIST-Response Evaluation Criteria in Solid Tumors; RP2D-M=recommended Phase II dose for ONC-392 as monotherapy; TEAE=treatment-emergent adverse event.

FIG. 2 shows a diagram of Part B Phase IA of the clinical trial of ONC-392 in combination with pembrolizumab. DLT=dose-limiting toxicity; NSCLC=non-small cell lung cancer; PD-(L)1=programmed cell death protein 1 or its ligand; Q3W=every 3 weeks; RECIST=Response Evaluation Criteria in Solid Tumors; RP2D-C=recommended Phase II dose for ONC-392 for combination therapy (ONC-392 plus pembrolizumab).

FIG. 3 shows a diagram of Part C Phase IB expansion of the trial of ONC-392 in monotherapy (upper) or in combination with pembrolizumab (lower). ALK=anaplastic lymphoma kinase; ECOG=Eastern Cooperative Oncology Group; EGFR=epidermal growth factor receptor; IO=immunotherapy; IV=intravenous; Mel=melanoma; mu=mutation; NSCLC=non-small cell lung cancer; TNBC=triple negative breast cancer; Q3W=every 3 weeks; RECIST v1.1=Response Evaluation Criteria in Solid Tumors version 1.1.

FIG. 4 shows the best overall response to ONC-392 monotherapy in Part A Phase IA of the clinical trial.

FIG. 5 shows the results of tumor tissue biomarker analysis of patients treated in Part A of an ONC-392 monotherapy clinical trial. The top panel shows the results in a NSCLC patient dosed at 3 mg/kg ONC-392 for 7 cycles, where CD8 (red), CD4 (green), Foxp3 (purple), and tumor cells (blue) are marked. The bottom left panel shows the pre-treatment results in an ovarian cancer patient, and the bottom right panel shows the results in an ovarian cancer patient treated with 10 mg/kg ONC-392 for 4 cycles, where CD8 (red), CD4 (green) Foxp3 (purple), and tumor cells (blue) are marked.

FIG. 6 shows goodness of fit for a final model.

FIG. 7 shows visual predictive check (VPC) Results.

FIG. 8 shows simulated PK profile at different dosing regimens: #1: 6 mg/kg, Q3W; #2: 10 mg/kg Q3W; #3: 10 mg/kg Q4W; #4 two loading doses of 10 mg/kg+6 mg/kg maintenance dose, Q3W. Solid line is the median pk profile; shaded area represents 90% prediction interval.

FIG. 9A-B shows model-predicted probability of ORR versus ONC-392 steady-state exposure using the highest dose in patients receiving ONC-392 monotherapy.

FIG. 10A-B shows model-predicted probability of Grade 3 or 4 TRAEs versus ONC-392 steadystate Cₘᵢₙ (FIG. 10A, top), Cₘₐₓ (FIG. 10A, bottom) and AUC (FIG. 10B) in patients receiving ONC-392 monotherapy.

FIG. 11A-B shows model-predicted probability of ORR versus ONC-392 steady-state exposure using the highest dose in patients with NSCLC (monotherapy).

FIG. 12A-B shows model-predicted probability of ORR and Grade ≥3 TRAEs versus ONC-392 steady-state exposure in patients with NSCLC.

### DETAILED DESCRIPTION

The inventors have discovered that the anti-CTLA-4 antibody dosing regimens described herein provide intrinsic lower toxicity and higher efficacy as compared to ipilimumab. Because of the improved safety the inventors have further determined that the clinical data on these regimens support prolonged dosing and clinical activity among cancer patients, including those with stage IV solid tumors. In particular, the anti-CTLA-4 antibodies that can be used in the dosing regimens disclosed herein are pH-sensitive forms that preserve CTLA-4 recycling and avoid lysosomal degradation, such as ONC-392.

### 1. Definitions.

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6,9, and 7.0 are explicitly contemplated.

### 2. Anti-CTLA-4 Antibody Dosing Regimens

Provided herein are anti-CTLA-4 antibody dosing regimens, which may be suitable for anti-CTLA-4 antibodies that exhibit pH-sensitive binding to CTLA-4 and avoid lysosomal degradation. In particular, the anti-CTLA-4 antibodies that may be used include pH-sensitive anti-CTLA-4 antibodies described in U.S. Pat. No. 10,618,960, the contents of which are incorporated herein by reference.

a. Anti-CTLA-4 Antibody

The anti-CTLA-4 antibody may comprise a light chain variable region comprising a complementarity determining region (CDR) 1 comprising the amino acid sequence RASENIYSNLA (SEQ ID NO: 1); a CDR2 comprising the amino acid sequence AATNLQS (SEQ ID NO: 2) (LC1), AATNLQD (SEQ ID NO: 3) (LC2), or AATSLQS (SEQ ID NO: 4) (LC3); and, a CDR3 comprising the amino acid sequence QHLWGTPYT (SEQ ID NO: 5).

The light chain variable region comprising LC1-LC3 may also comprise one of the following sequences, respectively:

LC1
(SEQ ID NO: 11)
DIQMTQSPSSLSASVGDRVTITCRASENIYSNLAWYQQKPGKAPKLLLY
AATNLQSGVPSRFSGSGSGTDFTLTISSLQPEDFATYYCQHLWGTPYTF
GGGTKLEIK
LC2
(SEQ ID NO: 12)
DIQMTQSPSSLSASVGDRVTITCRASENIYSNLAWYQQKQGKAPKLLLY
AATNLQDGVPSRFSGSGSGTDYTLTISSLQPEDFATYFCQHLWGTPYTF
GQGTKLEIK
LC3
(SEQ ID NO: 13)
DIQMTQSPSSLSASVGDRVTITCRASENIYSNLAWYQQKPGKAPKLLIY
AATSLQSGVPSRFSGSGSGTDFTLTISSLQPEDFATYYCQHLWGTPYTF
GGGTKVEIK

More particularly, the light chain comprising LC1-LC3 may comprise one of the following amino acid sequences, respectively:

LC1
(SEQ ID NO: 22)
DIQMTQSPSSLSASVGDRVTITCRASENIYSNLAWYQQKPGKAPKLLLY
AATNLQSGVPSRFSGSGSGTDFTLTISSLQPEDFATYYCQHLWGTPYTF
GGGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC*
LC2
(SEQ ID NO: 23)
DIQMTQSPSSLSASVGDRVTITCRASENIYSNLAWYQQKQGKAPKLLLY
AATNLQDGVPSRFSGSGSGTDYTLTISSLQPEDFATYFCQHLWGTPYTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC*
LC3
(SEQ ID NO: 24)
DIQMTQSPSSLSASVGDRVTITCRASENIYSNLAWYQQKPGKAPKLLIY
AATSLQSGVPSRFSGSGSGTDFTLTISSLQPEDFATYYCQHLWGTPYTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC*.

The anti-CTLA-4 antibody may comprise a heavy chain variable region comprising a CDR1 comprising the amino acid sequence GFSLTSYGLS (SEQ ID NO: 6); a CDR2 comprising the amino acid sequence YIWYDGNTNFHPSLKSR (SEQ ID NO: 7) (HC1), YIWYDGNTNFHSSLKSR (SEQ ID NO: 8) (HC2); or, YIWYDGNTNFHSPLKSR (SEQ ID NO: 9) (HC3); and, a CDR3 comprising the amino acid sequence TEGHYYGSNYGYYALDY (SEQ ID NO: 10).

The heavy chain variable regions comprising HC1-HC3 may comprise one of the following amino acid sequences, respectively:

HC1
(SEQ ID NO: 14)
QVQLQESGPGLVKPSETLSLTCTVSGFSLTSYGLSWIRQPPGKGLEWIG
YIWYDGNTNFHPSLKSRVTISKDTSKNQFSLKLSSVTAADTAVYYCAKT
EGHYYGSNYGYYALDYWGQGTSVTVSS
HC2
(SEQ ID NO: 15)
QVQLQESGPGLVKPSETLSLTCTVSGFSLTSYGLSWIRQPPGKGLEWIG
YIWYDGNTNFHSSLKSRVTISKDTSKSQVSLKLSSVTAADTAVYYCAKT
EGHYYGSNYGYYALDYWGQGTLVTVSS
HC3
(SEQ ID NO: 16)
QVQLQESGPGLVKPSETLSLTCTVSGFSLTSYGLSWIRQPPGKGLEWIG
YIWYDGNTNFHSPLKSRVTISVDTSKNQFSLKLSSVTAADTAVYYCAKT
EGHYYGSNYGYYALDYWGQGTLVTVSS

The anti-CTLA-4 antibody may comprise a heavy chain constant region comprising the amino acid sequence:

(SEQ ID NO: 17)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPG

The heavy chain constant region may also comprise one or more mutations. Relative to the sequence set forth in SEQ ID NO: 17, the one or more mutations may be selected from M135Y, S137T, T139E, S181A, E216A, and K217A, and a combination thereof. In one example, the heavy chain constant region of the antibody comprises all six mutations. The mutant heavy chain constant region may comprise the amino acid sequence:

(SEQ ID NO: 18)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLYITREPEVTCVVV
DVSHEDPEVKFNWYVDGVEVHNAKTKPREEQYNATYRVVSVLTVLHQDW
LNGKEYKCKVSNKALPAPIAATISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPG

Even more specifically, the heavy chain of the anti-CTLA-4 antibody comprising heavy chain variable regions HC1-HC3 may comprise one of the following amino acid sequences, respectively:

HC1
(SEQ ID NO: 19)
QVQLQESGPGLVKPSETLSLTCTVSGFSLTSYGLSWIRQPPGKGLEWIG
YIWYDGNTNFHPSLKSRVTISKDTSKNQFSLKLSSVTAADTAVYYCAKT
EGHYYGSNYGYYALDYWGQGTSVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGG
PSVFLFPPKPKDTLYITREPEVTCVVVDVSHEDPEVKFNWYVDGVEVHN
AKTKPREEQYNATYRVVSVLTVLHQDWLNGKEYKCKVSNKALPAPIAAT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPG**
HC2
(SEQ ID NO: 20)
QVQLQESGPGLVKPSETLSLTCTVSGFSLTSYGLSWIRQPPGKGLEWIG
YIWYDGNTNFHSSLKSRVTISKDTSKSQVSLKLSSVTAADTAVYYCAKT
EGHYYGSNYGYYALDYWGQGTLVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGG
PSVFLFPPKPKDTLYITREPEVTCVVVDVSHEDPEVKFNWYVDGVEVHN
AKTKPREEQYNATYRVVSVLTVLHQDWLNGKEYKCKVSNKALPAPIAAT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPG**
HC3
(SEQ ID NO: 21)
QVQLQESGPGLVKPSETLSLTCTVSGFSLTSYGLSWIRQPPGKGLEWIG
YIWYDGNTNFHSPLKSRVTISVDTSKNQFSLKLSSVTAADTAVYYCAKT
EGHYYGSNYGYYALDYWGQGTLVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGG
PSVFLFPPKPKDTLYITREPEVTCVVVDVSHEDPEVKFNWYVDGVEVHN
AKTKPREEQYNATYRVVSVLTVLHQDWLNGKEYKCKVSNKALPAPIAAT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPG**

A C-terminal lysine (K) may be additionally included in the amino acid sequence of the heavy chains set forth in SEQ ID NOs: 19-21, which may increase expression levels. The terminal lysine may be cleaved naturally during production of the anti-CTLA-4 antibody, or upon administration of the antibody.

pp 4637 (LC2/HC3): In one example the anti-CTLA-4 antibody comprises a light chain variable region comprising a CDR1 comprising the sequence set forth in SEQ ID NO: 1, a CDR2 comprising the sequence set forth in SEQ ID NO: 3, and a CDR3 comprising the sequence set forth in SEQ ID NO: 5. The heavy chain variable region comprises a CDR1 comprising the sequence set forth in SEQ ID NO: 6, a CDR2 comprising the sequence set forth in SEQ ID NO: 9, and a CDR3 comprising the sequence set forth in SEQ ID NO: 10. In particular, the light chain variable region may comprise the sequence set forth in SEQ ID NO: 12 and the heavy chain variable region may comprise the sequence set forth in SEQ ID NO: 16. More particularly, the light chain may comprise the sequence set forth in SEQ ID NO: 23, and the heavy chain may comprise the sequence set forth in SEQ ID NO: 21. This antibody may be referred to as ONC-392.

PP4631 (LC2/HC1): In another example, the anti-CTLA-4 antibody comprises a light chain variable region comprising a CDR1 comprising the sequence set forth in SEQ ID NO: 1, a CDR2 comprising the sequence set forth in SEQ ID NO: 3, and a CDR3 comprising the sequence set forth in SEQ ID NO: 5. The heavy chain variable region comprises a CDR1 comprising the sequence set forth in SEQ ID NO: 6, a CDR2 comprising the sequence set forth in SEQ ID NO: 7, and a CDR3 comprising the sequence set forth in SEQ ID NO: 10. In particular, the light chain variable region may comprise the sequence set forth in SEQ ID NO: 13 and the heavy chain variable region may comprise the sequence set forth in SEQ ID NO: 14. More particularly, the light chain may comprise the sequence set forth in SEQ ID NO: 23, and the heavy chain may comprise the sequence set forth in SEQ ID NO: 19.

PP4638 (LC3/HC3): In a further example, the anti-CTLA-4 antibody comprises a light chain variable region comprising a CDR1 comprising the sequence set forth in SEQ ID NO: 1, a CDR2 comprising the sequence set forth in SEQ ID NO: 4, and a CDR3 comprising the sequence set forth in SEQ ID NO: 5. The heavy chain variable region comprises a CDR1 comprising the sequence set forth in SEQ ID NO: 6, a CDR2 comprising the sequence set forth in SEQ ID NO: 9, and a CDR3 comprising the sequence set forth in SEQ ID NO: 10. In particular, the light chain variable region may comprise the sequence set forth in SEQ ID NO: 12 and the heavy chain variable region may comprise the sequence set forth in SEQ ID NO: 16. More particularly, the light chain may comprise the sequence set forth in SEQ ID NO: 24, and the heavy chain may comprise the sequence set forth in SEQ ID NO: 21.

b. Dosing Regimens

The anti-CTLA-4 antibody may be administered to a subject, which may be a human. The administration may be to treat a cancer, as described further herein. The anti-CTLA-4 antibody may be administered systemically, which may be via injection or intravenous administration. The antibody may be administered as a monotherapy, or as a combination therapy. The dosing regimen may comprise administering one or more doses of the anti-CTLA-4 antibody. Independently, each dose may be about 0.1 mg/kg, 0.3 mg/kg, 1 mg/kg, 3 mg/kg, 5, mg/kg, 6 mg/kg, 10 mg/kg, 15 mg/kg, 20 mg/kg, 25 mg/kg, 30 mg/kg, 50 mg/kg, or 100 mg/kg, or an amount in a range of two of these amounts. The dosing regimen may comprise periodic dosing, in which one of the foregoing doses is administered to the subject. At each cycle of dosing, the dose may be different from a previous dose. The dosing may involve escalating doses. In one example, the anti-CTLA-4 antibody is administered about every 1, 2, 3, 4, 5, or 6 weeks. In particular, the antibody is administered about every 3 weeks. When describing the period of a dosing cycle, “about” may mean ±1, 2, or 3 days.

In particular, the dose of the anti-CTLA-4 antibody may be about 1, 3, 6, or 10 mg/kg, or an amount in a range of two of these amounts. The dosing regimen may also comprise 10 mg/kg for two doses, followed by 1-6 mg/kg of extended dosing (that is, each subsequent dose is 1-6 mg/kg). The extended dosing may comprise administering a dose of 3 mg/kg or 6 mg/kg. In one example, each administration is once about every 3 weeks. The dosing may take place over a period of 3, 6, 9, 12, 15, 18, 21, 24, 27, 30, 33, 36, 39, 42, 45, 48 or 51 weeks. In one example, the anti-CTLA-4 antibody is used as a monotherapy and the dose is 10 mg/kg administered every 4 weeks. In a further example, the anti-CTLA-4 antibody is used in combination with pembrolizumab and the anti-CTLA-4 antibody dose is 3 or 6 mg/kg administered every 3 weeks, which may be coincident with the pembrolizumab dose schedule.

In another example, the pharmacokinetics of the anti-CTLA-4 antibody is monitored, and the dosing is adjusted adaptively to maintain a Cₘₐₓ of about 200-300 μg/mL. The Cₘₐₓ may be maintained for 6-52 weeks. The concentration may be measured from a subject's blood sample, which may be serum or plasma sample. In one example, the dosing is adjusted adaptively to maintain a Cₘₐₓ of about 200-300 μg/mL, about 225-250 μg/mL, about 225 μg/mL, or about 250 μg/mL. The dosing may be adjusted to avoid an excessively high Cₘₐₓ, which may be 200, 225, 250, or 300 μg/mL, particularly 250 μg/mL or 300 μg/mL. In one example, a deescalating dose level is administered to the subject if the Cₘₐₓ is excessively high, if the subject experiences one or more limiting toxicities, or if the subject has cancer and the subject achieves partial or complete response by Response Evaluation Criteria in Solid Tumors (RECIST) 1.1 criteria.

The anti-CTLA-4 antibody may be administered in combination, either separately or mixed with, a second therapeutic agent. The therapeutic agent may be an anti-cancer agent. In one example, the anti-cancer agent is administered on the same day as the anti-CTLA-4 antibody. In particular, the anti-cancer agent may be an anti-PD-1 or anti-PD-L1 antibody. In a specific example, the anti-cancer agent is pembrolizumab (KEYTRUDA). In one example, pembrolizumab is administered at 200 mg/cycle, every 21 days (3 weeks). In a further example, the second therapeutic agent is administered on the same day as the anti-CTLA-4 antibody.

c. Formulations

The anti-CTLA-4 antibody may be formulated at a dose described herein. In one example, the formulation comprises 1, 2, 3, 4, 5, 6, 7, 8, 9, 10, 15, 20, 25, 30, 50, or 100 mg/mL of the anti-CTLA-4 antibody, or an amount in a range thereof. In one example, the amount is 5 mg/mL. The formulation may comprise 5, 10, 15, 20, 25, 30, 35, or 40 mM histidine buffer, or an amount in a range of two of these amounts. In one example, the amount is 20 mM. The formulation may also comprise 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0% (w/v) α, α-trehalose dihydrate, or an amount in a range of two of these amounts. In one example, the amount is 8.8%. The formulation may comprise 0.01, 0.02, 0.03, 0.04, 0.05, 0.06, 0.07, 0.08, 0.09, or 0.10 (w/v) polysorbate 80, or an amount in a range of two of these amounts. In one example, the amount is 0.06%. The formulation may of a pH of 5.5, 5.6, 5.7, 5.8, 5.9, 6.0, 6.1, 6.2, 6.3, 6.4, or 6.5, or a pH in a range thereof. Equivalent ingredients to histidine buffer, α, α-trehalose dihydrate, and polysorbate 80 for formulating antibodies are known in the art, and may also be used as substitutes.

### 3. Cancer Treatments

The compositions and dosing regimens therefor may be used to treat cancer. Provided herein is a method of treating cancer in a subject in need thereof, which may comprise administering an anti-CTLA-4 antibody described herein to the subject. Also provided herein are the anti-CTLA-4 antibody for use in treating cancer, and use of the anti-CTLA-4 antibody in the manufacture of a medicament for treating cancer. The method, use, or medicament may comprise administering the anti-CTLA-4 antibody or medicament using a dosing regimen described herein.

The cancer may be a solid tumor. The cancer may be one of progressive locally advanced and metastatic cancer. The cancer may be stage IV cancer. The subject may exhibit failure or intolerance to standard of care guidelines, which may be National Comprehensive Cancer Network Clinical Practice Guidelines in Oncology (NCCN Guidelines). The cancer may be refractory or resistant to anti-PD-1/PD-L1 treatment. The resistance may be primary resistance or acquired resistance with disease progression after immunotherapy. The primary PD-1 resistance may be defined as disease progression within 24 weeks of initiation of anti-PD-(L)1 therapy. The acquired PD-1 resistance may be defined as 24 weeks or more of disease control (CR, PR or SD) after initiation of anti-PD-(L)1 therapy and has subsequently progressed after 24 weeks. The cancer may be immunotherapy naïve, and may be PD-L1 positive, such as by having PD-L1 Tumor Proportion Score≥1%. The cancer may be non-small cell lung cancer. In another example, the cancer is ovarian, cervical, gastroesophageal, lung, or ovarian cancer.

The subject may be 18 years of age or older. The subject may have metastatic disease or locally advanced disease not amenable to local therapy. The subject may also have failed established standard medical anti-cancer therapies, which may be other than pembrolizumab for a given tumor type, or may have been intolerant to such therapy. The subject may have an Eastern Cooperative Oncology Group performance status of ≤2.

The cancer may be a neoplasm or tumor resulting from abnormal uncontrolled growth of cells. The cancer may be a leukemia or lymphoma. The cancer may also involve cells that have the potential to metastasize to distal sites.

The cancer may be one of the following: a carcinoma, such as that of the bladder, breast, colon, kidney, liver, lung, ovary, pancreas, stomach, cervix, thyroid or skin; squamous cell carcinoma; a hematopoietic tumor of lymphoid lineage, such as a leukemia, acute lymphocytic leukemia, acute lymphoblastic leukemia, B-cell lymphoma, T-cell lymphoma, or Berkitt's lymphoma; a hematopoietic tumor of myeloid lineage, such as acute and chronic myelogenous leukemia or promyelocytic leukemia; a tumor of mesenchymal origin, such as fibrosarcoma or rhabdomyoscarcoma; a tumor such as melanoma, seminoma, tetratocarcinoma, neuroblastoma, or glioma; a tumor of the central and peripheral nervous system, such as astrocytoma, neuroblastoma, glioma, or schwannoma; a tumor of mesenchymal origin, such as fibrosarcoma, rhabdomyosarcoma, or osteosarcoma; or a tumor such melanoma, xenoderma pegmentosum, keratoactanthoma, seminoma, thyroid follicular cancer, or teratocarcinoma.

The subject who has cancer may have a histologically or cytologically confirmed diagnosis of solid tumors and progressive locally advanced or metastatic disease, and may have failure or intolerance to established standard medical anti-cancer therapies per standard of care guidelines, which may be NCCN Guidelines. The tumors may be of a type for which pembrolizumab has been approved as standard of care treatment. The subject who has cancer may have advanced or metastatic cancer, and may have disease progression after prior systemic cancer treatments. In one example, the cancer is pancreatic cancer, triple negative breast cancer, non-small cell lung cancer (NSCLC) with Epidermal Growth Factor mutation or other targetable mutations, PD-1-refractory NSCLC, head and neck cancer, and ovarian cancer.

The subject with cancer may have advanced/metastatic cancer, and may be treatment naïve, immunotherapy (IO) naïve, or refractory/resistant (R/R) to anti-programmed cell death protein 1 or its ligand (anti-PD-(L)1. The cancer may be NSCLC IO naïve, PD-L1-positive with a Tumor Proportion Score (TPS)≥1%, NSCLC IO R/R, melanoma IO naïve, or melanoma IO R/R. The cancer may be recurrent and/or metastatic (R/M) adenoid cystic carcinoma, which may not be amenable to curative intent surgery or radiation.

The cancer may be a melanoma, metastatic melanoma, PD (L)-1-refractory melanoma, non-small cell lung adenocarcinoma, metastatic NSCLC, NSCLC with driver mutations (for example, EGFR/ALK mutations or other targetable mutations), PD-1-refractory NSCLC, head and neck cancer, adenoid cystic carcinoma (which may be R/M), squamous carcinoma, triple negative (basal-type) breast cancer, pancreatic cancer, renal cell carcinoma, cervical cancer, endometrial cancer, colon cancer, hepatocellular carcinoma, other solid tumors, or metastatic colorectal cancer (which may have microsatellite instability).

The cancer may be caused by aberrations in apoptosis. The cancer may be a follicular lymphomas, a carcinoma with one or more p53 mutations, a hormone-dependent tumor of the breast, prostate or ovary, a precancerous lesion such as familial adenomatous polyposis, or a myelodysplastic syndrome. The cancer may be a malignancy or dysproliferative change (such as metaplasia or dysplasia), or a hyperproliferative disorders, and may be in the ovary, bladder, breast, colon, lung, skin, pancreas, or uterus. In particular, the cancer may also be sarcoma, melanoma, or leukemia.

The present invention has multiple aspects, illustrated by the following non-limiting examples.

### Example 1

### Anti-CTLA-4 Antibody Treatment Safety and Efficacy

This example demonstrates safety and efficacy of the anti-CTLA-4 antibody ONC-392 for treating cancer, particularly advanced solid tumors and non-small cell lung cancer (NSCLC).

### Indication

For Part A Phase IA ONC-392 monotherapy dose-finding cohorts, patients with a histologically or cytologically confirmed diagnosis of solid tumors who have progressive locally advanced or metastatic disease after failure of or intolerance to established standard medical anti-cancer therapies, as per standard of care guidelines, such as NCCN guidelines, will be enrolled.

For Part B Phase IA combination dose finding cohorts, patients with a histologically or cytologically confirmed diagnosis of solid tumors who have progressive locally advanced or metastatic disease, and the tumor types are cancers that pembrolizumab has been approved as standard of care treatment, will be enrolled. Treatment naïve, or checkpoint inhibitor immunotherapy naïve or refractory/resistant patients can be enrolled.

For Part C Phase IB ONC-392 monotherapy cohorts, patients with advanced/metastatic cancers who have disease progression after prior systemic treatments will be enrolled in the following monotherapy arms: pancreatic cancer (Arm A), triple negative breast cancer (TNBC) (Arm B), non-small cell lung cancer (NSCLC) with EGFR (Epidermal Growth Factor Receptor) mutation or other targetable mutations (Arm C), PD-1 refractory NSCLC (Arm I), head and neck cancers (Arm K), ovarian cancer (Arm L), solid tumors that are not eligible to or other tumor types than those specified in Arm A, B, C, I, K, and L (Arm M).

For Part C Phase IB combination therapy cohorts, patients with advanced/metastatic cancers who are treatment naïve, or immunotherapy (IO)-naïve, or refractory/resistant (R/R) to anti-programmed cell death protein 1 or its ligand (anti-PD-(L)1) treatment will be enrolled in following combination therapy arms: NSCLC IO naïve, PD-L1-positive with PD-L1 Tumor Proportion Score (TPS)≥1% (Arm D), NSCLC IO R/R (Arm E, regardless of PD-L1 status), melanoma (Mel) IO naïve (Arm F); and Mel IO R/R (Arm G).

### Summary of the Study Design

The study consists of three linked parts:

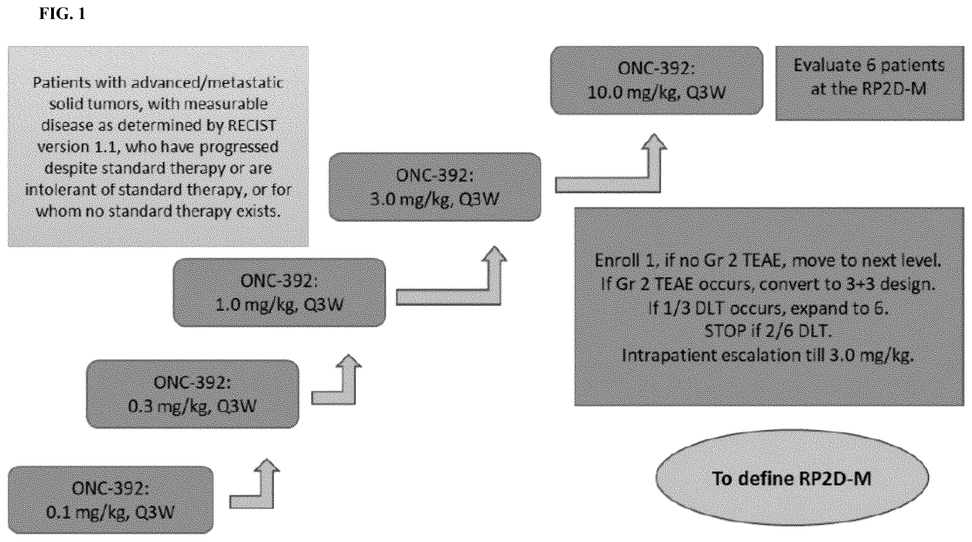

Part A (FIG. 1) is a dose-finding rapid titration study of ONC-392 as a single agent in patients with advanced solid tumors of various histology. The aim of this trial is to define the recommended Phase II dose for ONC-392 monotherapy (RP2D-M).

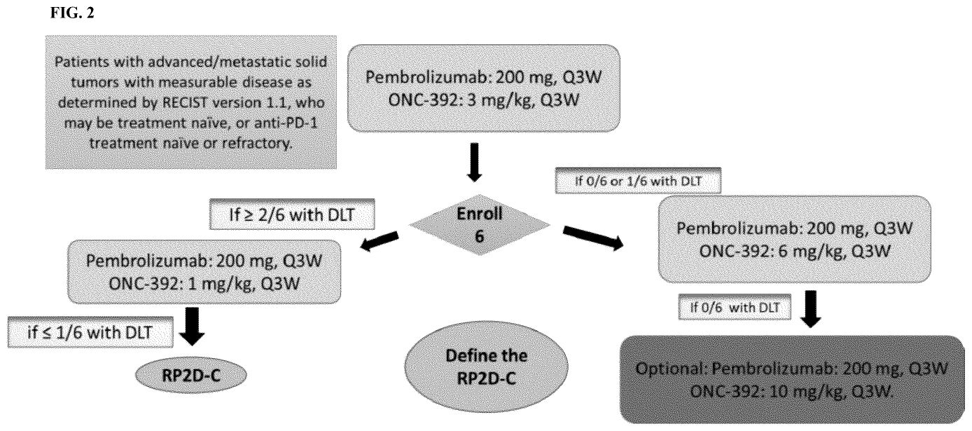

Part B (FIG. 2) is a dose-finding study of ONC-392 in combination with a standard dose of 200 mg Pembrolizumab to define the recommended Phase II dose for ONC 392 in combination with Pembrolizumab (RP2D-C) in patients with advanced solid tumors of various histology for which Pembrolizumab is approved as standard of care (SOC).

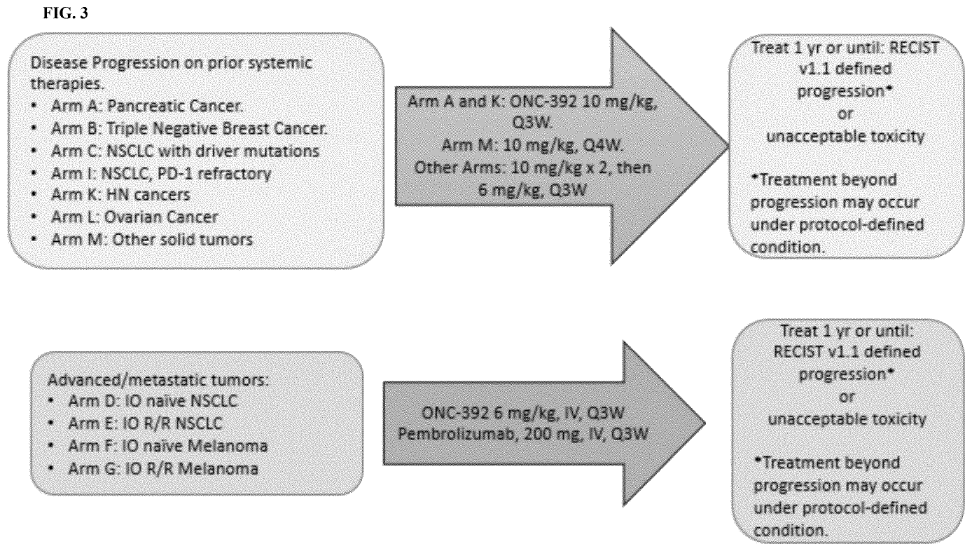

Part C (FIG. 3) Phase IB expansion cohorts of ONC-392 in monotherapy and in combination therapy with Pembrolizumab to determine safety and initial efficacy. Additional arms may be included in future protocol amendments. Arms A, B, C, I, K, L, M monotherapy expansion cohorts can be initiated after the RP2D-M is determined. Arms D-G expansion cohorts with combination therapy can be initiated after the RP2D-C is determined.

Arm A: Pancreatic Cancer Cohort, ONC-392 monotherapy, will enroll advanced/metastatic pancreatic cancer patients, including ampullary cancer, who have progressive disease after first and second lines of systemic treatment.

Arm B: TNBC Cohort, ONC-392 monotherapy, will enroll advanced/metastatic TNBC patients who have progressive disease after prior systemic treatments, including checkpoint inhibitor immunotherapy.

Arm C: NSCLC Mono Cohort 1, ONC-392 monotherapy, will enroll advanced/metastatic NSCLC patients with EGFR or ALK mutations or other targetable mutations who have progressive disease after prior systemic treatments, including targeted therapy or checkpoint inhibitors.

Arm D: NSCLC IO Naïve Cohort, ONC-392/Pembrolizumab combination therapy, will enroll advanced/metastatic NSCLC cancer patients who are treatment naïve, or anti PD(L)1 immunotherapy naïve and PD-L1-positive (PD L1 TPS≥1%).

Arm E: NSCLC IO R/R Cohort, ONC-392/Pembrolizumab combination therapy, will enroll advanced/metastatic NSCLC cancer patients who are R/R to prior anti-PD-(L)1 immunotherapy regardless of PD-L1 status.

Arm F: Melanoma IO Naïve Cohort, ONC-392/Pembrolizumab combination therapy, will enroll advanced/metastatic Melanoma patients who are treatment naïve, or checkpoint inhibitor immunotherapy naive. Prior systemic chemotherapy or targeted therapy are allowed.

Arm G: Melanoma IO R/R Cohort, ONC-392/Pembrolizumab combination therapy, will enroll advanced/metastatic melanoma patients who are R/R to anti-PD-(L)1 immunotherapy.

Arm I: NSCLC Mono Cohort 2, ONC-392 monotherapy, will enroll advanced/metastatic NSCLC patients without EGFR or ALK mutations or other targetable mutations who have progressive disease after prior systemic treatments, including chemotherapy or checkpoint inhibitors. Patient must have anti-PD-(L)1 treatment, either alone or in combination, as last treatment before enrollment. Prior anti-CTLA-4 treatment is allowed.

Arm K: Head and Neck Cancer, ONC-392 monotherapy, will enroll advanced/metastatic Squamous Cell Carcinoma (HNSCC) and other histology types except adenoid cystic carcinoma, with or without positive HPV, who have progressive disease after prior systemic treatments, including chemotherapy or checkpoint inhibitors, or immunotherapy.

Arm L: Ovarian Cancer, ONC-392 monotherapy, will enroll patients with advanced/metastatic ovarian cancer, including primary peritoneal cancer and fallopian tube cancer, who have progressive disease after prior systemic treatments, including chemotherapy, targeted therapy or checkpoint inhibitors.

Arm M: Solid Tumors, ONC-392 monotherapy, will enroll patients with advanced/metastatic solid tumors who are not eligible for above mentioned monotherapy arms, who have progressive disease after prior systemic treatments, including chemotherapy, targeted therapy or checkpoint inhibitors.

### Objectives and Endpoints

TABLE 1
Objective and endpoints

| Part | Purpose | Objectives | Endpoints |
|---|---|---|---|
| Part A | Primary | Dose-finding rapid titration | Maximum tolerated dose (MTD) or Recommended |
| | | | Phase II dose for ONC-392 monotherapy (RP2D-M) |
| | | Safety | Incidence of Treatment Emergent Adverse Events |
| | | | (TEAEs) |
| | Secondary | Characterize the | PK parameters |
| | | pharmacokinetic (PK) profile | |
| | | of single agent ONC-392 | |
| | Exploratory | Efficacy | Objective response rate (ORR) and duration of |
| | | | response (DoR), disease control rate (DCR), best |
| | | | overall response (BoR) as assessed by Investigators |
| | | | based on Response Evaluation Criteria in Solid Tumors |
| | | | version 1.1 (RECIST 1.1) and immune RECIST |
| | | | (iRECIST). |
| | | | Progression-free survival (PFS) as assessed by |
| | | | Investigators based on RECIST 1.1 and immune |
| | | | RECIST (iRECIST). |
| | | | Overall survival (OS) following administration of |
| | | | ONC-392. |
| Part B | Primary | Dose-finding | Recommended Phase II dose for ONC-392 in |
| | | | combination with a standard dose of pembrolizumab |
| | | | (RP2D-C) for Part B1. |
| | | Safety | Incidence of Treatment Emergent Adverse Events |
| | | | (TEAEs) |
| | Secondary | Characterize the PK profile of | PK parameters. |
| | | ONC-392 in combination with | |
| | | standard of care (SOC) | |
| | | Pembrolizumab. | |
| | Exploratory | Efficacy | Objective response rate (ORR) and duration of |
| | | | response (DoR), disease control rate (DCR), best |
| | | | overall response (BoR) as assessed by Blinded |
| | | | Independent Central Review (BICR) based on |
| | | | Response Evaluation Criteria in Solid Tumors version |
| | | | 1.1 (RECIST 1.1) and immune RECIST (iRECIST). |
| | | | Progression-free survival (PFS) as assessed by BICR |
| | | | based on RECIST 1.1 and immune RECIST |
| | | | (iRECIST). |
| | | | Overall survival (OS) following administration of |
| | | | ONC-392. |
| Part C | Primary | Efficacy | ORR as assessed by BICR based on RECIST 1.1 after |
| | | | the patient receives first ONC-392 treatment either as |
| | | | monotherapy or as combination therapy with |
| | | | pembrolizumab. |
| | | Safety | Incidence of TEAEs |
| | Secondary | Efficacy parameters | ORR, DoR, BoR and DCR. |
| | Exploratory | Additional Efficacy | PFS as assessed by the Investigator based on RECIST |
| | | parameters: | 1.1 and iRECIST. |
| | | | OS following administration of ONC-392. |
| | | | ORR, DoR, BoR and disease control rate (DCR) as |
| | | | assessed by the BICR based on iRECIST. |
| | | Exposure-response correlation | PK parameters |

### Key Study Eligibility Criteria

To be eligible for the study, patients had to be 18 years of age or older, had to have metastatic disease or locally advanced disease not amenable to local therapy, and had to have failed established standard medical anti-cancer therapies other than pembrolizumab for a given tumor type, or have been intolerant to such therapy, or in the opinion of the Investigator have been considered ineligible for a particular form of standard therapy on medical grounds. Patients must have had an Eastern Cooperative Oncology Group (ECOG) performance status of ≤2. The table below shows the study eligibility criteria.

TABLE 2
Study eligibility

| Inclusion Criteria | | Exclusion Criteria | |
|---|---|---|---|
| 1. | Age ≥18 yrs old. | 1. | Patients who have not recovered to NCI CTCAE ≤1 from an adverse event |
| 2. | Male or Female; Female must have | | (AE) due to cancer therapeutics except the chemotherapy-associated |
| | negative pregnancy test. | | peripheral neuropathy (motor or sensory) or endocrine AE that has |
| 3. | Must have ECOG score ≤1. | | recovered to CTCAE ≤2 will be allowed. The washout period for cancer |
| 4. | A histological or cytological diagnosis | | therapeutic drugs should be 21 days for chemotherapy, radiation, or targeted |
| | of solid tumors and progressive | | therapy or 28 days for monoclonal antibody therapy. Best supportive care, |
| | metastatic disease or progressive locally | | such as thyroxine, insulin, steroid replacement treatment, blood transfusion |
| | advanced disease | | and therapy for non-cancer conditions are allowed. |
| 5. | Must have measurable target lesion | 2. | Patients who are currently enrolled in any other clinical trial testing an |
| | according to RECIST V1.1. | | investigational agent or device, or with concurrent other systemic cancer |
| 6. | Adequate organ function as determined | | therapeutics. |
| | by laboratory tests | 3. | Patients who are on chronic systemic steroid therapy at doses higher than |
| 7. | Voluntary agreement to participate as | | 10 mg/day prednisone or equivalent within 7 days before first treatment. |
| | evidenced by written informed consent. | 4. | Patients who have active brain metastases or leptomeningeal metastases. |
| 8. | Female patient: agreement on | 5. | Patients who have an active infection requiring systemic IV antibiotics |
| | contraceptive methods. | | within 14 days prior to administration of ONC-392 or combined ONC-392 |
| 9. | Male patient: agreement on | | and Pembrolizumab. Regular treatment of urinary tract infection (UTI) |
| | contraceptive methods. | | and/or topical treatment are allowed. |
| 10. | In expansion cohort arms, patient | 6. | Patients who, in the opinion of the treating Investigator, have a history or |
| | must agree to grant study team the | | current evidence of any condition, therapy, or laboratory abnormality that |
| | access to archival diagnostic tissue | | might confound the results of the study, interfere with the patient's |
| | (recut slides or tumor biopsy). | | participation for the full duration of the study, or make study participation |
| | | | not in the best interest of the patient. Investigator should discuss with |
| | | | Sponsor. |
| | | 7. | Patients with known psychiatric or substance abuse disorders that in the |
| | | | opinion of the investigator, would interfere with cooperation with the |
| | | | requirements of the trial. |
| | | 8. | Patients who are pregnant or breastfeeding. |
| | | 9. | For Part B, patients who are deemed to be not suitable for Pembrolizumab as |
| | | | standard of care treatment. |

### Dosage/Dosage Form, Route, and Dose Regimen

For dose escalation in monotherapy, five dose levels of ONC-392 will be evaluated: 0.1 mg/kg, 0.3 mg/kg, 1.0 mg/kg, 3.0 mg/kg and 10 mg/kg. ONC-392 will be administered as an IV infusion over a minimum of 30 minutes for dose levels of 0.1, 0.3, and 1.0 mg/kg and a minimum of 60 minutes for the 3.0 mg/kg dose level. At the 10 mg/kg dose level, a minimum of 90 minutes of infusion time is required for the first dose, and a minimum of 60 minutes for subsequent doses. The ONC-392 dosing interval will be 21 days (every 3 weeks [Q3W]). Intrapatient dose escalation up to 3 mg/kg is allowed.

For the combination of ONC-392 and Pembrolizumab, ONC-392 will be administered first as an IV infusion over a minimum of 60 minutes except that the first dose of ONC-392 10 mg/kg should be administrated over a minimum of 90 minutes. For the 6.0 mg/kg ONC-392 dose level, the IV infusion should be given over 60 minutes. Pembrolizumab will then be administered IV over a minimum of 30 minutes at a fixed 200 mg/dose. There will be a gap of at least 30 minutes between the end of the ONC-392 infusion and the start of the Pembrolizumab infusion. ONC-392 and Pembrolizumab should not be mixed during administration. ONC-392 and Pembrolizumab will both be given Q3W.

Study treatment (both monotherapy and combination therapy) may be continued for 4 additional cycles (optional) after a patient has confirmed progressive disease (PD) based on immune Response Evaluation Criteria in Solid Tumors (iRECIST) if the patient tolerates the treatment.

Study treatment (both monotherapy and combination therapy) should be stopped for unacceptable toxicity, voluntary withdrawal by the patient, or at 1 year (13- or 17 cycles), whichever occurs first (refer to Section 5.7 for options after 1 year).

In Part A and Part B, the administration of ONC-392, either as a single agent or in combination with Pembrolizumab, will require the monitoring of vital signs and electrocardiograms (ECGs) as indicated in Table 3.

Part A: ONC-392, 5 dose levels (0.1 mg/kg, 0.3 mg/kg, 1.0 mg/kg, 3.0 mg/kg, and 10.0 mg/kg) by IV infusion Q3W. Intrapatient dose escalation allowed to 3.0 mg/kg. Dose finding until RP2D-M. Up to a total of 17 doses in 12 months.

Part B: ONC-392+Pembrolizumab at 200 mg/dose by IV infusion Q3W. Dose finding until RP2D-C. Up to a total of 17 cycles in 12 months.

Part C: Arms A-C and Arms I, K, L, M. ONC-392 at the RP2D-M by IV infusion according to following dosing schedule. The treatment period will be up to one year.

Part C: Arms D-G. ONC-392 at RP2D-C+Pembrolizumab at 200 mg by IV infusion Q3W. Up to a total of 17 cycles in 12 months. The ONC-392 RP2D-C has been determined to be 6 mg/kg.

### Number of Patients Planned

Part A: A minimum of 10 and a maximum of 30 patients will be enrolled in ONC-392 monotherapy to identify the RP2D-M.

Part B: A minimum of 6 and a maximum of 36 patients will be enrolled in ONC-392 and Pembrolizumab combination therapy to identify the RP2D-C. Dose de-escalation will stop if more than 2 DLTs out of 6 patients at 1 mg/kg.

Part C: The expansion cohort study will be carried out with an adaptive trial design. For each cohort, a futility stopping rule will apply. A minimum of 15 and a maximum of 30 patients will be enrolled in each expansion arm except for arm A, which a maximum of 30 efficacy evaluable patients will be enrolled.

### Part A: ONC-392 Single Agent

The Part A Phase IA trial was tested up to five predefined dose levels: 0.1 mg/kg, 0.3 mg/kg, 1 mg/kg, 3 mg/kg and 10 mg/kg of ONC-392 as monotherapy through IV infusion every 21 days (Q3W). The trial used an accelerated titration design. Intra-patient dose escalation was tested in the first patient receiving 0.1 mg/kg, 0.3 mg/kg, 1.0 mg/kg without any AE. This patient was escalated to 3.0 mg/kg and received 3 cycles at this dose without any AE. The second patient started at 0.3 mg/kg without any AE. The enrollment was then converted to a 3+3 design at 3.0 mg/kg and 10.0 mg/kg levels in the protocol below.

### Part B: Combination of ONC-392 and Pembrolizumab in NSCLC

Part B was designed as a Phase IA dose escalation/de-escalation study followed by a Phase IB expansion component at the RP2D-C for the combination of ONC-392 with pembrolizumab in two cohorts of patients with NSCLC.

The dose for pembrolizumab was fixed at 200 mg/cycle dosed every 21 days (Q3W).

### Part B Phase IA Study

The Phase IA study was started at the dose one level below the RP2D-M dose for ONC-392 with 200 mg of pembrolizumab and was to initially enroll 6 patients. The ONC-392 dose was to be adjusted according to following scenario:

(1) If 1/6 patients develop a dose limiting toxicity (DLT), then the dose one level below the RP2D-M were to be declared as RP2D-C.

Or:

(2) If 0/6 patients develop a DLT, 6 additional patients were to be enrolled at the RP2D-M dose level for ONC-392. If ≤1/6 of the additional patients developed a DLT, then the RP2D-M were to be declared as PR2D-C.

Or:

(3) When 2 DLTs occur before 6 patients were enrolled, the ONC-392 dose was to be de-escalated to the next dose level until ≤1/6 patients treated at that dose developed a DLT. This dose level was designated RP2D-C. If dose level 1 (0.1 mg/kg) was too toxic using the above rule, further exploration of the combination of the drugs was to be stopped.

### Part B Phase IB Study

The Part B Phase IB expansion cohorts were both designed for patients with advanced NSCLC and included an immunotherapy naïve cohort and a refractory/resistant cohort. The six patients treated at the RP2D-C in Part B Phase IA, were evaluable for efficacy. One of the aims of the expansion cohorts was to arrive at a more comprehensive safety profile for the combination of ONC-392 at the RP2D-C plus pembrolizumab. To ensure the safety of the patients enrolled in the two expansion cohorts, a Pocock-type boundary was used to allow early stopping for excess toxicity at any given time. The trial was to be stopped at any point in time if the incidence of DLTs was significantly higher than 0-20%.

In the anti-PD(L)1 immunotherapy naïve population, patients with advanced NSCLC with PD-L1-positive (PD-L1 TPS≥1% or otherwise indicated for pembrolizumab) were to be included in the study. 18 subjects were to be enrolled for the Phase IB expansion cohort.

In the anti-PD(L)1 refractory/resistant population, patients with advanced NSCLC who had disease progression or did not tolerate anti-PD(L)1 containing treatment (including monotherapy or combination therapy, or immunotherapy combined with chemotherapy) after 4 or more cycles were to be included in this study. Prior CTLA-4 therapy was allowed. Prior history of irAE but recovered was allowed. 18 subjects were to be enrolled for the Phase IB expansion cohort.

The response rates for PD-(L)1 therapy naïve and refractory/resistant cohorts was to be determined separately at 6 months after the first treatment.

### Safety Results of Part A

The demographics of the patients evaluated in Part A of the ONC-392 trial are shown in the following table.

TABLE 3

| Category | Number |
|---|---|
| Patients | 10 |
| Gender (F/M) | 7/3 |
| White/Asian/Black | 6/3/1 |
| Median age (range) | 62 (43-81) |
| Cancer type | n/stage |
| NSCLC | 4/IV |
| Ovarian cancer | 4/IV |
| GE junction cancer | 1/IV |
| Cervical cancer | 1/IV |

The following table shows a summary of treatment-emergent adverse events (TEAEs).

TABLE 4

| | Any TEAEs | | | Treatment-Related TEAEs | | |
|---|---|---|---|---|---|---|
| | 3 mg/kg | 10 mg/kg | Total | 3 mg/kg | 10 mg/kg | Total |
| | (N = 4) | (N = 6) | (N = 10) | (N = 4) | (N = 6) | (N = 10) |
| Any TEAEs | 4 (100%) | 6 (100%) | 10 (100%) | 2 (50%) | 6 (100%) | 8 (80%) |
| Gr1/2 | 4 (100%) | 6 (100%) | 10 (100%) | 2 (50%) | 5 (83%) | 7 (70%) |
| Gr3/4* | 1 (25%) | 4 (67%) | 5 (50%) | 0 | 3 (50%) | 3 (30%) |
| Gr5 | 0 | 0 | 0 | 0 | 0 | 0 |
| DLT | 0 | 0 | 0 | 0 | 0 | 0 |

*Three irAEs are pancreatitis (1) and colitis (2) after 3 or 4 cycles of 10 mg/kg treatment

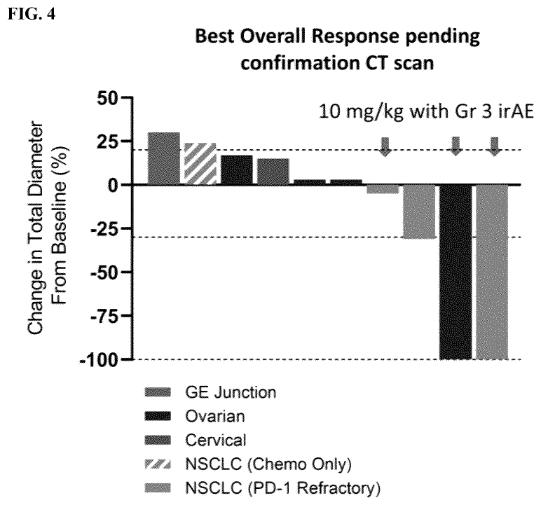

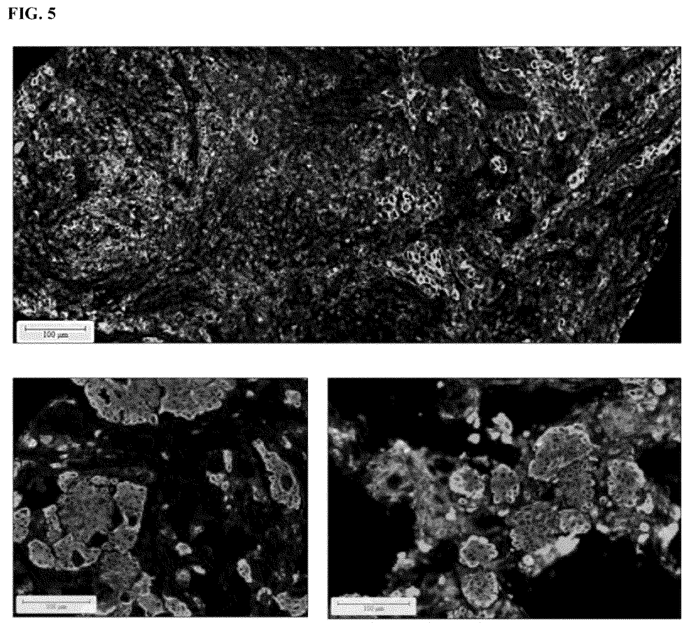

These results indicate that ONC-392 at both doses was generally well-tolerated. The Grade 3 irAEs of pancreatitis and colitis were manageable and reversible. PR2D for monotherapy was 10 mg/kg, Q3W. The best response to ONC-392 monotherapy is shown in FIG. 4. FIG. 5 shows the results of tumor tissue biomarker analysis. The top panel shows the results in a NSCLC patient dosed at 3 mg/kg ONC-392 for 7 cycles, where CD8, CD4, Foxp3, and tumor cells are marked respectively by red, green, magenta, and cyan colors. The bottom left panel shows the pre-treatment results in an ovarian cancer patient, and the bottom right panel shows the results in an ovarian cancer patient treated with 10 mg/kg ONC-392 for 4 cycles, where CD8, CD4, Foxp3, and tumor cells are marked respectively by red, green, magenta, and cyan colors.

The results show that ONC-392 was well tolerated. The longest dosing was 3 mg/kg for up to 11 cycles. No DLT or Grade 3/4 AEs occurred during the DLT observation period at any dose. The maximum tolerable dose was not reached. The recommended phase 2 dose for monotherapy was determined to be 10 mg/kg. The following Grade 3/4 AEs occurred in three patients after 3 or 4 cycles of treatment at 10 mg/kg ONC-392: colitis/hypokalemia (2) and pancreatitis (1). Two of these three patients had unconfirmed complete response, and one had stable disease with shrinking tumor burden. Other drug-related AEs were grade 1/2, and those that occurred in more than two patients included infusion-related reactions, pruritis, fatigue, and TSH increase.

### Clinical Results

In addition, beneficial activity was observed in 6/10 patients. Two of 6 patients treated at 10 mg/kg ONC-392 exhibited complete response, two of 6 patients treated at 10 mg/kg ONC-392 had stable disease with a significant reduction of tumor burden or a biomarker of enhanced T cell activation in the tumor, and two out of 4 patients treated at 3 mg/kg had stable disease (SD) at greater than 7 months. Stable disease was observed in 7 out of 10 patients, and partial response was observed in 1 out of 10 patients in the first tumor assessment. Further, clinical improvements were observed among three PD-(L)1 refractory/resistant patients with NSCLC (one with complete response; one with disease control at greater than 24 weeks who became eligible for surgery; and, one with stable disease at 8 weeks with continued treatment).

### Safety and Efficacy Conclusion

ONC-392 was generally safe and well tolerated. Treatment-related AEs could be managed. And the maximum tolerable dose was not reached at the 10 mg/kg dose. ONC-392 also demonstrated therapeutic anti-tumor activities. As the first pH-sensitive monoclonal antibody that preserves CTLA-4 recycling and avoids lysosomal degradation, ONC-392 may fundamentally change the risk/benefit ratio of CTLA-4 targeting by conferring improved efficacy and reduced toxicity.

### Example 2

### Clinical Safety and Efficacy Results for Anti-CTLA-4 Antibody Administered at Various Doses

This example demonstrates safety and efficacy of ONC-392 administered at 10 mg/kg Q3W for two cycles, followed by 6 mg/kg Q3W for up to 12 months. This regimen is selected based on the efficacy and safety information, PK, and exposure-response analyses of an ongoing ONC-392 study.

### Clinical Safety and Efficacy Results of ONC-392 Monotherapy

The following 4 dosing regimens of ONC-392 monotherapy were tested:
1) 6 mg/kg Q3W for melanoma cohort (Arm J);
2) 10 mg/kg Q3W in pancreatic cancer cohort and HNSCC cohort (Arms A and K);
3) 10 mg/kg Q4W in advanced solid tumor cohort (Arm M); and
4) 10 mg/kg Q3W×2, followed by 6 mg/kg Q3W in PD-1/PD-L1-resistant NSCLC cohort and ovarian cancer cohort (Arms I and L).

Safety data of all 153 patients who received ONC-392 monotherapy with different treatment regimens tested appeared safe and generally tolerated. Table 5 presents the summary of safety and ORR in NSCLC patients receiving ONC-392 monotherapy. The 8 patients in Regiment #3 (10 mg/kg Q4W) were NSCLC patients who have PD-1/PD-L1 inhibitor therapy followed by chemotherapy. They had disease progression on chemotherapy prior to enrollment in the study. Two of them are continuing in treatment and none had tumor response in this group of patients. The 34 patients receiving ONC-392 in Regimen #4, which is the proposed treatment dosing regimen, came from two arms, 12 patients with driver mutations in Arm C and 22 patients with PD-1/PD-L1 resistant NSCLC in Arm I. The efficacy of tumor response was observed in PD-1/PD-L1 resistant NSCLC patients.

As shown in Table 5, comparison of safety data for regimens #2, #3 and #4 in NSCLC patients, regimen #4 (10 mg/kg Q3W×2, followed by 6 mg/kg Q3W) had lowest incidence of grade≥3 TRAEs (12%), treatment-related SAEs (12%), and TRAEs leading to study treatment discontinuation (6%).

TABLE 5
Summary of TEAEs and ORR in NSCLC Patients Receiving ONC-392 Monotherapy (Part A and Part C)

| | | Regimen #2 | Regimen #3 | Regimen #4 | |
|---|---|---|---|---|---|
| | | 10 mg/kg | 10 mg/kg | 10 mg/kg × | |
| | 3 mg/kg | Q3w | Q4W | 2 + 6 mg/kg | Total |
| | (N = 1) | (N = 3) | (N = 8) | (N = 34) | (N = 46) |
| Any Grade TEAEs | 1 (100.0%) | 3 (100.0%) | 6 (75.0%) | 28 (82.3%) | 38 (82.6%) |
| ≥G3 TEAEs | 0 | 2 (66.7%) | 4 (50.0%) | 20 (58.8%) | 26 (56.5%) |
| Any Grade TRAEs related to ONC-392 | 0 | 2 (66.7%) | 4 (50.0%) | 18 (52.9%) | 24 (52.1%) |
| ≥G3 TRAEs | 0 | 2 (66.7%) | 2 (25.0%) | 4 (11.8%) | 8 (17.4%) |
| Any SAEs | 0 | 2 (66.7%) | 4 (50.0%) | 18 (52.9%) | 24 (52.1%) |
| SAEs Related to ONC-392 | 0 | 2 (66.7%) | 2 (25.0%) | 4 (11.8%) | 8 (17.4%) |
| TRAEs leading to Dose Interruption | 0 | 0 | 2 (25.0%) | 4 (11.8%) | 6 (13.0%) |
| TRAEs leading to Dose Reduction | 0 | 0 | 0 | 0 | 0 |
| TRAEs leading to Permanent | 0 | 1 (33.3%) | 1 (12.5%) | 2 (5.9%) | 4 (8.7%) |
| Discontinuation | | | | | |
| Death Related to ONC-392 | 0 | 0 | 0 | 0 | 0 |
| ORR in Evaluable Patients | 0/1 = 0% | 1/3 = 33% | 0/6 = 0% | 3/14 = 21.4% | 4/24 = 16.7% |

The low rate of severe TRAE and clinical activity support regimen #4 for the proposed indication. This dose selection is further supported by additional clinical pharmacology analyses outlined below.

### Population Pharmacokinetic Results

A population PK model was constructed with 420 measurable PK observations from 70 patients, including 57 patients receiving monotherapy of ONC-392 and 13 patients receiving combination therapy with pembrolizumab.

### Methods

### Population PK

### Data Source

As the cutoff date of Jul. 8, 2022, the PK data of ONC-392 covered doses ranging from 0.1 to 10 mg/kg administered by the intravenous (IV) route. The dataset contains 446 PK samples from 71 patients with various cancer types. Twenty-six PK samples were excluded from the analysis due to one of the following reasons: 1) outlier, 2) pre-dose samples with negative time since first dose, 3) missing or potentially incorrect sampling time records or dosing information (Subject 001-130). A population PK model was constructed with 420 measurable PK observations from 70 patients, including 57 patients receiving monotherapy of ONC-392 and 13 patients receiving combination therapy with pembrolizumab.

### Software and Method

Nonlinear Mixed Effects Modeling software (NONMEM® version 7.4; ICON, Hanover, MD, US), a software package for nonlinear mixed-effects analysis, was used for population PK modeling and simulations to derive exposure metrics for the subsequent E-R analysis. R (version 4.0.1) was used for diagnostic plots and visual check of all plots.

Nonlinear mixed-effects models were fitted to the concentration-time data of ONC-392 as a function of dose, time and other subject-level covariates. One or two compartment structure model; linear or empirical target mediated drug disposition (TMDD) models were tested.

Covariate-parameter relationships were initially evaluated graphically, followed by direct testing of potential covariates in the population PK model. Model evolution was based on goodness of fit (GOF) plots, objective function value (OFV), precision and plausibility of PK parameter estimation, and visual predictive check (VPC).

### Summary of Baseline Covariates

Summaries of baseline continuous and categorical covariates are provided in Table 6 and Table 7.

TABLE 6
Summary of Baseline Continuous Covariates

| | Mean (SD) | |
|---|---|---|
| Covariate | Median (Min-Max) | |
| Age (years) | 63.05 | (9.24) |
| | 63.00 | (43.00-83.00) |
| Body weight (kg) | 74.73 | (17.20) |
| | 73.10 | (44.50-130.00) |
| Albumin (g/dL) | 3.59 | (0.52) |
| | 3.70 | (1.80-4.50) |
| AST (U/L) | 31.70 | (27.37) |
| | 21.00 | (10.00-141.00) |
| Bilirubin (μmol/L) | 0.61 | (0.35) |
| | 0.50 | (0.10-2.00) |
| Creatinine clearance (mL/min) | 87.30 | (40.39 |
| | 80.45 | (25.35-271.25) |

TABLE 7
Summary of Baseline Categorical Covariates

| Covariate | Number of Subjects (%) | |
|---|---|---|
| Sex | | |
| Male | 26 | (37.10%) |
| Female | 44 | (62.90%) |
| Race | | |
| White/Caucasian | 57 | (81.40%) |
| Black/African American | 4 | (5.71%) |
| Asian | 6 | (8.57%) |
| Other/unknown | 3 | (4.29%) |
| Cancer Type | | |
| Non-small Cell Lung Cancer | 28 | (28.60%) |
| Malignant Melanoma | 6 | (8.57%) |
| Hepatocellular Carcinoma | 2 | (2.86%) |
| Head and Neck Carcinoma | 3 | (4.29%) |
| Ovarian Carcinoma | 17 | (24.30%) |
| Colorectal Cancer | 2 | (2.86%) |
| Sarcoma | 1 | (1.43%) |
| Pancreatic Cancer | 4 | (5.71%) |
| Triple Negative Breast Cancer | 2 | (2.86%) |
| Other | 13 | (18.60%) |

### Population PK Results

PK of ONC-392 are best described by a 2-compartment model with first-order elimination. The systemic clearance (CL) of ONC-392 was estimated to be 182 mL/day (Table 8), and the terminal t_{1/2} was estimated to be 25.7 days. Baseline albumin was identified as a significant covariate for CL; increased albumin level is associated with decreased CL. Body weight was identified as a significant covariate for volume terms, including central volume (V1) and peripheral volume (V2); increased body weight is associated with increased V1 and V2. No effects of age, sex, race, AST, bilirubin, creatinine clearance, or cancer type on ONC-392 PK were detected. None of these covariates were considered clinically significant relevant. Of note, concurrent chemotherapy with PD-1 was not a significant PK covariate.

TABLE 8
PK Parameters of Final Model

| | Fixed Effects | | IIV | | Shrinkage |
|---|---|---|---|---|---|
| Parameter | Estimate | RSE % | Estimate | RSE % | (%) |
| CL (mL/day) | 182 | 4.1% | 0.0234 | 41.2% | 44.9% |
| Q (mL/day) | 637 | 10.9% | — | — | — |
| V1 (mL) | 2850 | 4.1% | 0.0414 | 17.3% | 11.4% |
| V2 (mL) | 3340 | 12.8% | 0.382 | 38.2% | 14.3% |
| | | | 0.114 (off- | 27.5% | |
| | | | diagonal | | |
| | | | covariance | | |
| | | | for V1 and V2) | | |
| ALB on CL | −0.866 | 31.6% | — | — | — |
| BW on V1 | 0.412 | 29.4% | — | — | — |
| and V2 | | | | | |
| Proportional | 0.152 | 3.0% | — | — | 10.4% |
| Residual | | | | | |
| Error (SD) | | | | | |

CL = systemic clearance; Q = inter-compartmental clearance; V1 = central volume; V2 = peripheral volume.

Sparse PK sampling at approximately peak and trough time was conducted in patients receiving one of the 4 treatment regimens in Part C cohort expansion of this clinical study.

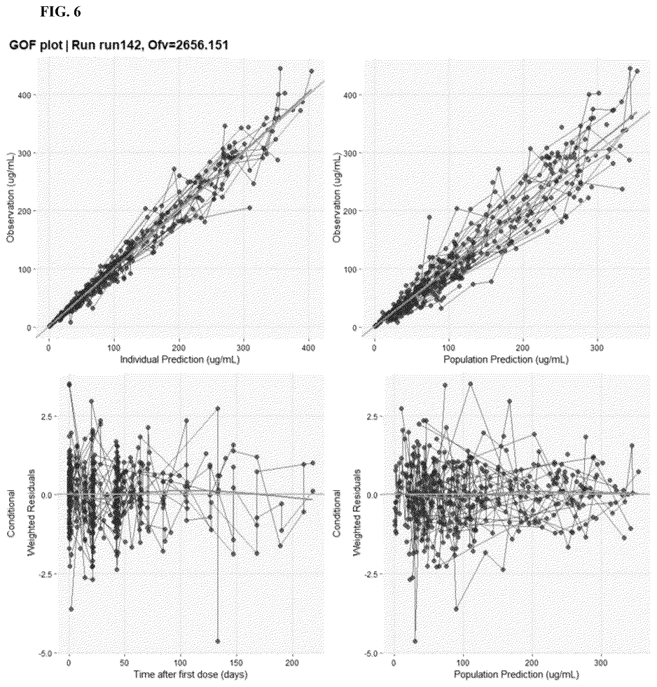

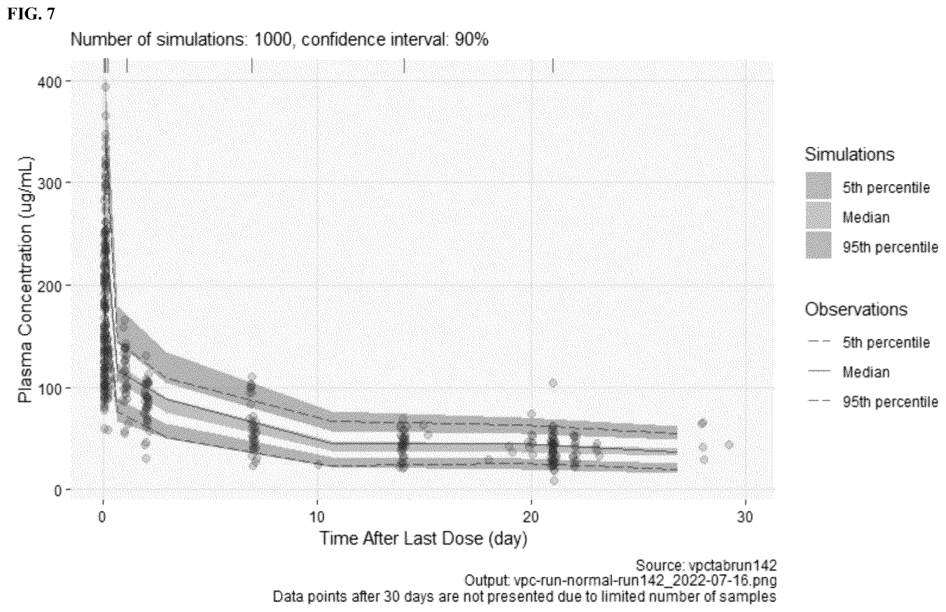

Goodness of Fit (GOF) of the final model showed reasonable model fitting and good agreement between observed and model predictions. The residual plots did not show any model mis-specification (FIG. 6). Visual Predictive Check (VPC) results showed adequate predictability of the final model (FIG. 7).

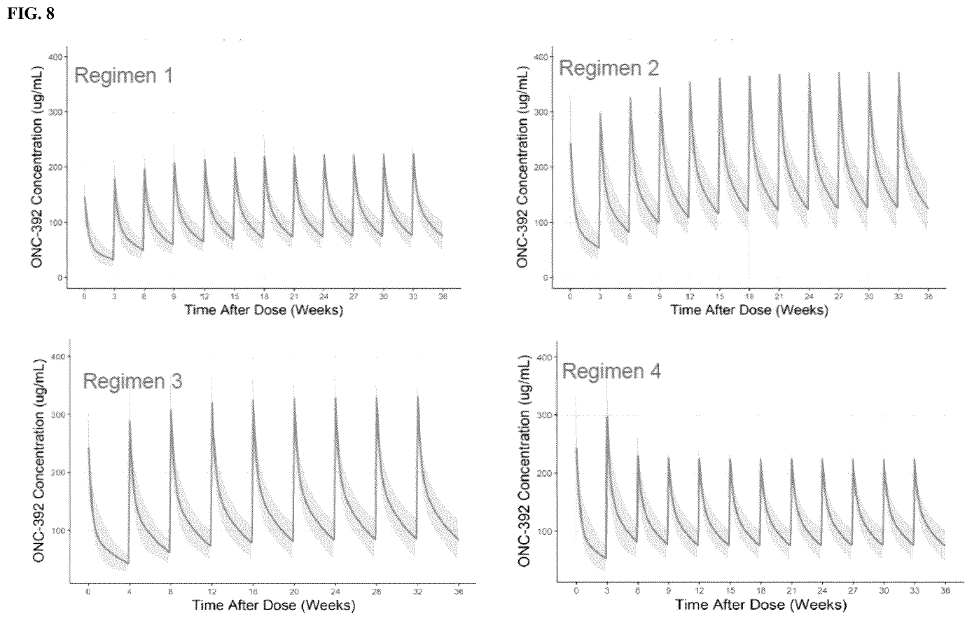

The observed PK profiles of ONC-392 at various dose levels indicated that the proposed dosing regimen of 10 mg/kg Q3W×2+6 mg/kg Q3W allows the systemic concentration to reach the steady state level after the 2nd dose and maintains high trough levels throughout the dosing period (FIG. 8), which are much higher than the Kd of ONC-392 (1.95 μg/mL, based on in vitro binding to human CTLA-4) and ensure adequate ONC-392 exposure in tumor environment to maximize the antitumor activity of ONC-392.

### Exposure-Response (E-R) Analyses

### Deriving Exposure Metrics

The final population PK model of ONC-392 was used to predict concentration-time profiles based on posterior Bayesian estimates. The model-simulated steady-state exposure was used as PK metrics to assess the preliminary relationship between exposure and efficacy/safety outcomes. Data from patients with available PK (N=70) from the preliminary results of Study ONC-392-001 are used for efficacy and safety analyses. Among the 70 patients with exposure data, 57 received monotherapy of ONC-392, of which 17 were NSCLC patients.

The data used for exposure-response (E-R) analyses is summarized in Table 9.

TABLE 9
Summary of Data Used for Exposure-Response Analyses

| Number of Patients | Monotherapy | Combination | Total |
|---|---|---|---|
| PK Analysis Population | 57 | 13 | 70 |
| Dose: 3 mg/kg | 4 | 7 | 11 |
| Dose: 6 mg/kg | 1 | 6 | 7 |
| Dose: 10 mg/kg | 52 | 0 | 52 |
| BOR | | | |
| CR | 3 (10 mg/kg) | 0 | 3 |
| PR | 5 (10 mg/kg) | 2 (3 mg/kg) | 7 |
| SD | 14 | 6 | 20 |
| PD | 14 | 2 | 16 |
| Missing | 21 | 3 | 24 |

Due to the limited number of patients, the results of this preliminary ER analysis should be interpreted with caution.

### Exposure-Response Relationship in Patients on ONC-392 Monotherapy

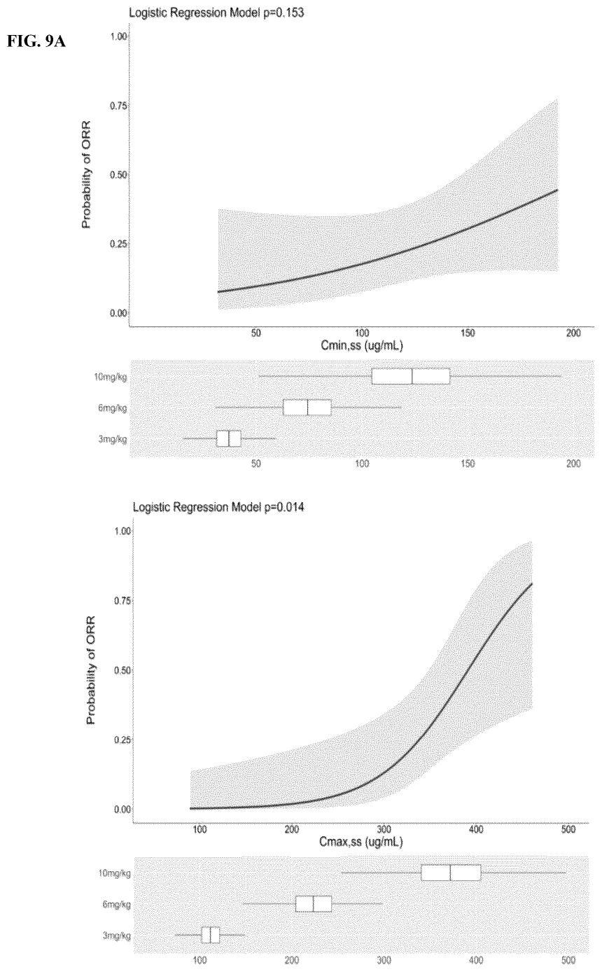

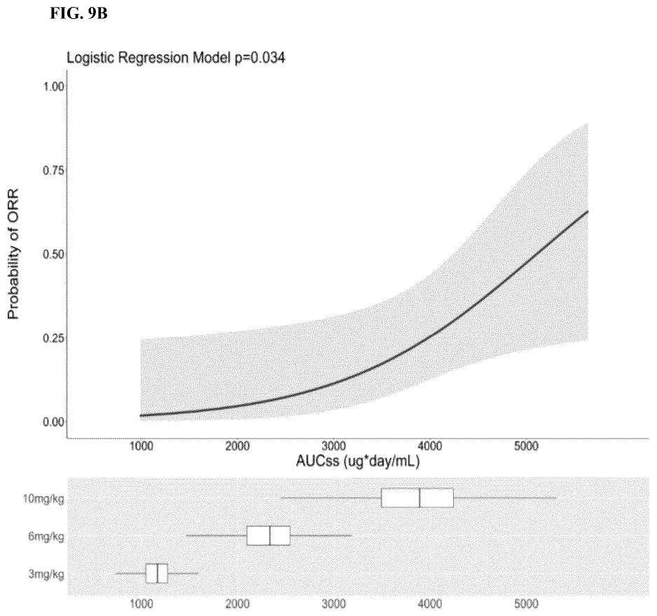

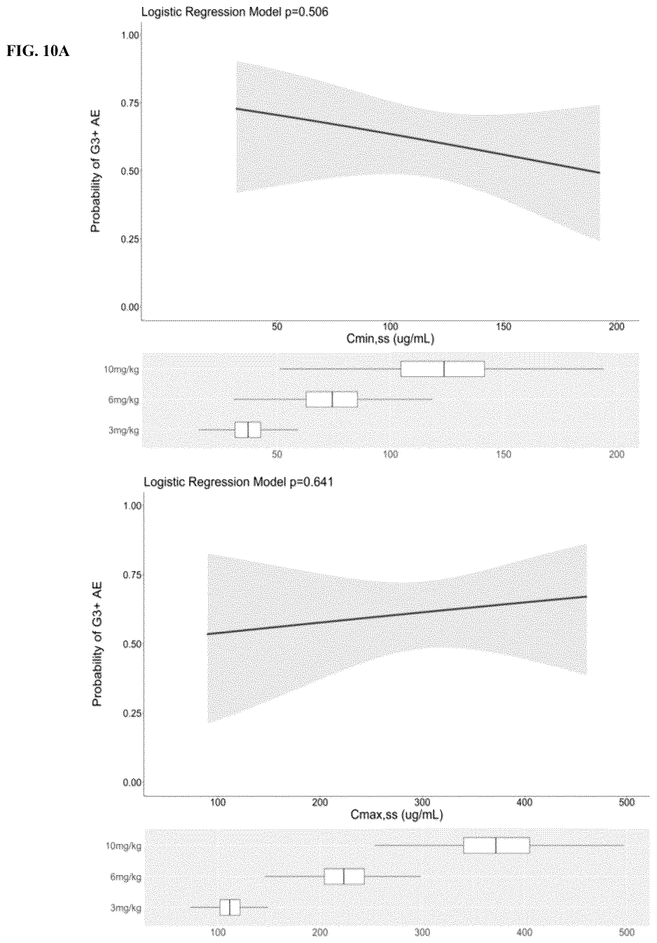

Tumor assessment data are available in 36 patients who received monotherapy and had PK data. 8 of them had clinical response (PR or CR). The probability of an ORR appears to be higher with an increased exposure (C_{min,ss}, C_{max,ss} and AUCₛₛ) (FIG. 9A-B). Such a correlation was the most significant for C_{max,ss}, consistent with the notion that higher Cₘₐₓ of ONC-392 is required for more efficient elimination of T regulatory cells in the tumor environment.

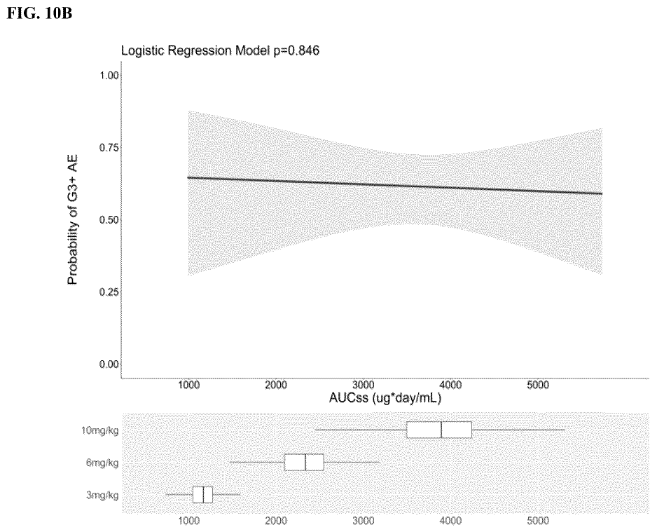

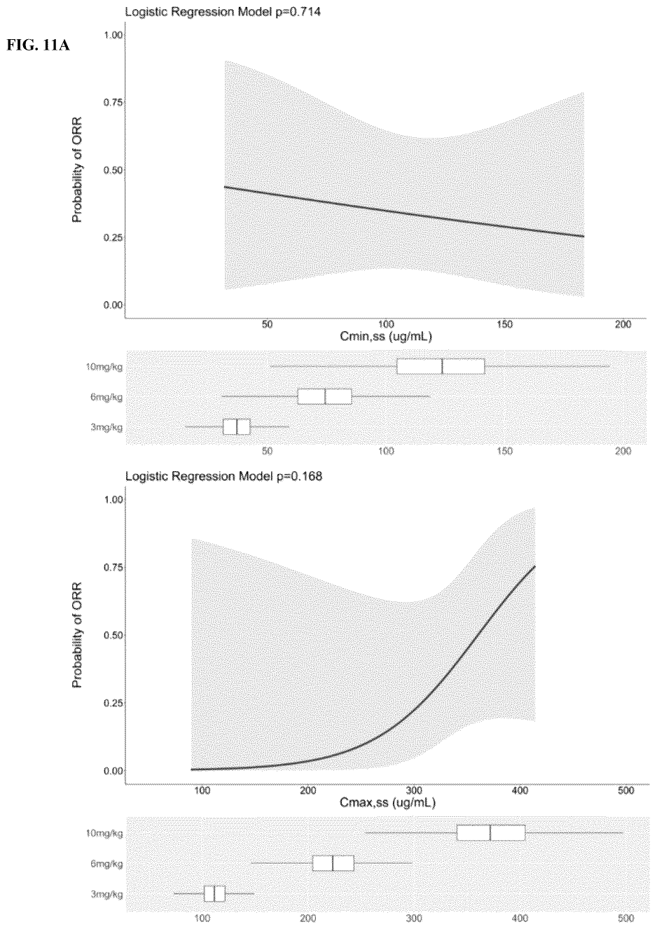

The exposure and safety relationship was also examined. Safety data are available in 57 patients who received ONC-392 monotherapy and had PK data. No clear correlation of frequency or severity of drug-related TEAEs with the steady-state exposure was observed, regardless which exposure parameters (C_{min,ss}, C_{max,ss} or AUCₛₛ) were tested (FIG. 10A-B), suggesting that exposure is not a determinant of severe TRAEs within the exposure range tested.

Exposure-Response Relationship in Patients with NSCLC

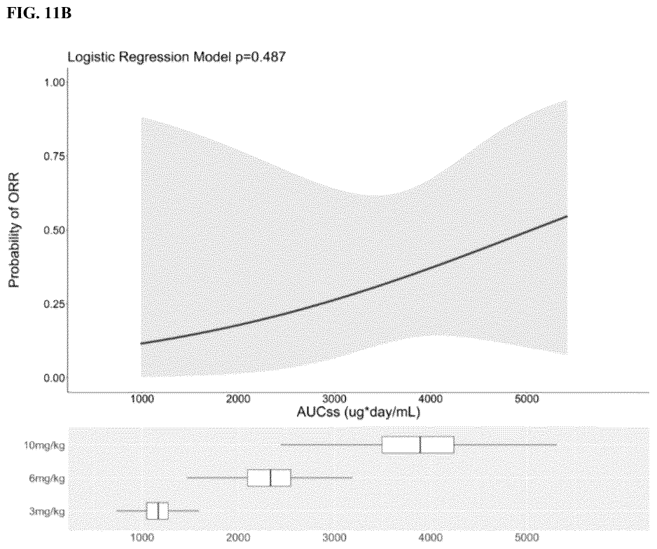

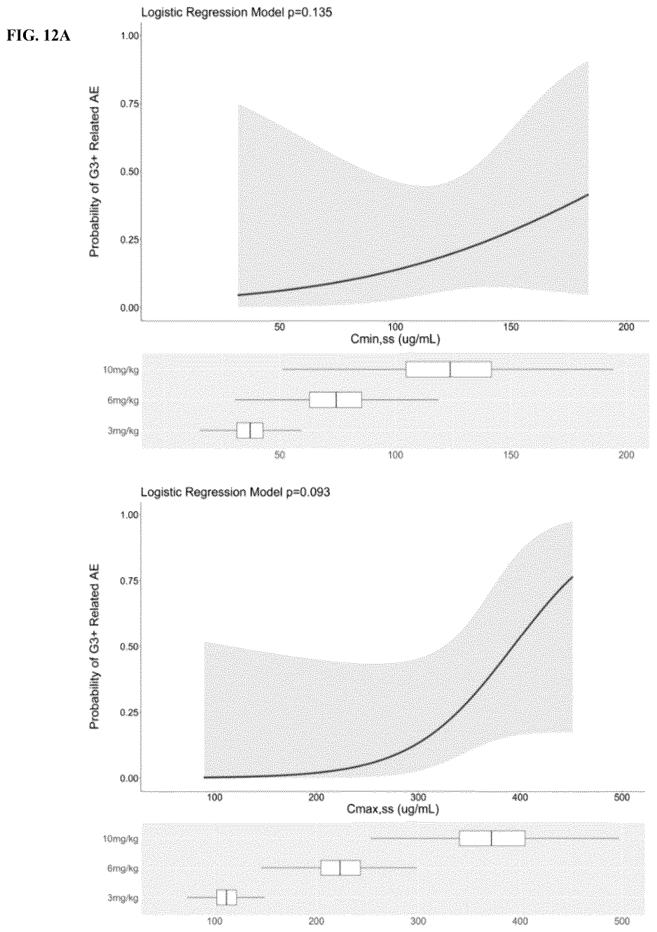

Tumor assessment data are available in 12 NSCLC patients who received monotherapy and had PK data. The exposure-response analyses showed no statistically significant relationship but suggested a trend, although not statistically significant, of higher the probability of an ORR with increasing exposure (Cₘₐₓ, ss) (FIG. 11A-B). A less clear trend was seen for AUCss, while no relationship was observed for C_{min,ss}.

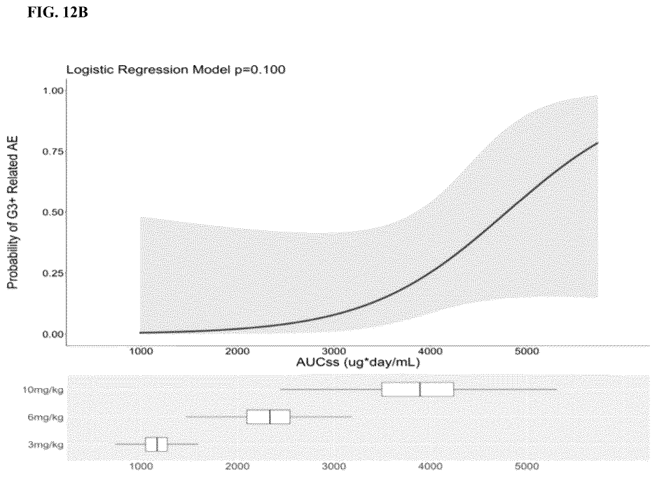

However, in the NSCLC patients (N=17), patients with higher steady-state exposure tend to have a higher probability of having grade ≥3 TRAEs (FIG. 12A-B). Comparing the safety data from patients in Arm I: PD(L)1-resistant NSCLC and Arm L: ovarian cancer who received the same ONC-392 regimen of 10 mg/kg Q3W×2, followed by 6 mg/kg Q3W, patients with NSCLC had a lower incidence of Grade≥3 TRAEs (13.0% Grade 3, 0% Grades 4-5) compared with the ovarian cancer cohort (40% grade 3, 3.3% Grade 4, 0% Grade 5, cutoff date of 31 Aug. 2022). The difference between data from NSCLC (FIGS. 12A-B) and those from the pooled 57 patients with different tumor types (FIG. 10A-B) is due to the higher rate of TRAE among those with C_{max,ss} less than 300 μg/mL in the pooled data set (e.g., patients with other cancer types), when compared with NSCLC patients with similarly lower exposure. The fact that NSCLC patients are less prone to developing severe TRAE at a lower exposure is consistent with the notion that higher exposure is required for development of TRAE in this population.

### SUMMARY

Sparse PK sampling at approximately peak and trough time was conducted in patients receiving one of the 4 treatment regimens in Part C cohort expansion of this clinical study. The observed PK profiles of ONC-392 at various dose levels indicated that the proposed dosing regimen of 10 mg/kg Q3W×2+6 mg/kg Q3W allows the systemic concentration to reach the steady state level after the 2nd dose and maintains the median trough levels which are nearly 40-fold higher than the Kd of ONCC-392 (1.95 μg/mL, based on in vitro binding to human CTLA-4).

Notably, preliminary efficacy data in patients with either the 10 mg/kg Q3W or the loading dose of 10 mg/kg+6 mg/kg maintenance dose Q3W are encouraging. All responders experienced PR or CR at the time of the first or second tumor assessment, with most responders identified at the first tumor assessment. This suggests early exposure could play a key role in anticancer treatment with ONC-392. A high loading dose of 10 mg/kg Q3W×2 was therefore chosen to ensure adequate ONC-392 exposure in tumors in early treatment cycles to maximize the antitumor activity of ONC-392. At the same time, a maintenance dose of 6 mg/kg Q3W will serve to maintain the efficacy and minimize the toxicities of ONC-392 for the long-term use.

It is of note that the no significant increase in probability of ORR was observed until the Cₘₐₓ reached around 225 μg/ml, a level that is immediately attenable by regimen #2-4, but barely attainable by regimen #1 used in dose expansion, and unattainable by the 3 mg/kg used in dose escalation.

In the NSCLC patients with both safety and PK data (N=17), patents with higher steady-state exposure (C_{max,ss}) also have a higher probability of having grade ≥3 TRAEs (FIG. 12). The fact that NSCLC patients are less prone to developing severe TRAE at a lower exposure suggest that regimen #4, which have lower maintenance dose may allow better patient safety over extended period.

In dose escalation and dose expansion parts of this clinical study, the safety and efficacy of ONC-392 monotherapy were evaluated in ˜130 patients in 5 different dose/dosing regimens with increasing levels of exposure, namely 3 mg/kg Q3W, 6 mg/kg Q3W, 10 mg/kg Q3W, 10 mg/kg Q4W and 10 mg/kg×2 followed by 6 mg/kg, Q3W. Based on the clinical results and pharmacology data, including exposure-response modeling results, 10 mg/kg×2 followed by 6 mg/kg, Q3W was considered as the optimal dose and chosen for the Phase 3 development in patients. The rationale for the dosing regimen is re-capitulated below.

The exposure/dose response suggest that the probability of ORR is best correlated with Cₘₐₓ, and that slope of rapidly increasing probability of ORR occurred after the Cₘₐₓ reached around 225 μg/ml, based on the inflection point of curve depicting the Cₘₐₓ-ORR relation. As shown in Table 10, this threshold is achievable immediately if the starting dose is 10 mg/kg but never achievable at 3 mg/kg Q3W. Indeed, at 3 mg/kg, no clinical response was observed among the 4 patients despite prolonged dosing of up to 9 cycles and the best safety profile. At 6 mg/kg Q3W, it takes 6 cycles to reach near this level. Since patients who failed systemic immune therapy had poor prognosis, it is unlikely that a regimen with nearly 4 months of delay in achieving potentially effective dose would confer meaningful clinical benefit.

TABLE 10
Projected Cₘₐₓ by treatment regimens

| Regimens | Cycle 1 | Cycle 2 | Cycle 3 | Cycle 4 | Cycle 5 | Cycle 6 |
|---|---|---|---|---|---|---|
| 3 mg/kg | 75.1 ± 15.6 | 90.7 ± 18.4 | 99.1 ± 18.4 | 104.0 ± 17.6 | 106.9 ± 16.9 | 108.8 ± 16.2 |
| Q3W (Part | | | | | | |
| A) | | | | | | |
| 6 mg/kg | 150.1 ± 31.2 | 181.3 ± 36.8 | 198.2 ± 36.7 | 208.0 ± 35.3 | 213.9 ± 33.7 | 217.6 ± 32.4 |
| Q3W | | | | | | |
| (Regimen | | | | | | |
| 1, Part C) | | | | | | |
| 10 mg/kg | 250.2 ± 52.0 | 302.2 ± 61.4 | 330.4 ± 61.2 | 346.6 ± 58.8 | 356.4 ± 56.2 | 362.6 ± 53.9 |
| Q3W | | | | | | |
| (Regimen | | | | | | |
| 3, Part C) | | | | | | |
| 10 mg/kg | 250.2 ± 52.0 | 292.2 ± 56.4 | 311.6 ± 54.1 | 321.4 ± 51.2 | 326.8 ± 49.0 | 329.8 ± 47.3 |
| Q4W | | | | | | |
| (Regimen | | | | | | |
| 3, Part C) | | | | | | |
| 6 mg/kg | 250.2 ± 52.0 | 302.2 ± 61.4 | 230.3 ± 41.4 | 225.7 ± 35.0 | 224.3 ± 32.2 | 224.0 ± 30.8 |
| Q3W | | | | | | |
| following | | | | | | |
| 10 mg/kg | | | | | | |
| Q3Wx2 | | | | | | |
| (Regimen | | | | | | |
| 4, Part C) | | | | | | |

Among regimens #2-4, which are projected to confer therapeutic activities, the selection of regimen #4 is based on clinical data and PK. Regimen 2, at 10 mg/kg Q3W, which gives the highest exposure, had clinical activities but also highest toxicities relative to other dose/regimens. Thus, among patients received 10 mg/kg Q3W, 50% (3/6) of the patients in Part A (dose escalation) and 39% (16/41) of the patients in Part C dose expansion of the study developed grade 3 or 4 TRAE. 2/2 PD(L)1-resistant NSCLC patients who were treated with this regimen developed Grade 3 TRAE. Thus, regimen was not chosen for safety concerns.

Of the other two regimens starting with 10 mg/kg, i.e., regimen #3, 10 mg/kg Q4W, and regimen #4, with loading dose 10 mg/kg Q3W×2, followed by 6 mg/kg Q3W maintenance, higher Cₘₐₓ is achieved earlier in the regimen #4 but more sustained exposure is delivered by regimen 3. Both regimens showed comparable clinical activities (ORR 13-14%) among all cancer types. It is of note that regimen 4 yielded an ORR of 30% and DCR of 70% among 10 evaluable patients at the first and second tumor assessments in patients with PD (L)-1-resistant NSCLC. These data suggest that 10 mg/kg Q3W×2 followed by 6 mg/kg dose can potentially provide meaningful clinical benefit. Based on the preliminary results from this clinical study, the tumor response occurred mostly at the first tumor assessment after 2 or 3 cycles, suggesting that first 2 doses of 10 mg/kg may be critical and required to achieve the response.

More importantly, the safety of regimen #4 for the proposed indication appears very favorable as only 4/34 (11.8%) NSCLC patients developed Grade 3 TRAE and no patients have developed grade 4 or 5 TRAE (Table 5). Based on the correlation between Cₘₐₓ and risks of severe TRAE among the NSCLC (FIG. 11A, lower curve graph and whisker plot and FIG. 12A lower curve graph and whisker plot), sustained high exposure delivered by regimen #3 may increase safety risk over long-run.

In conclusion, the preliminary exposure-response analysis results and clinical safety and efficacy findings suggest that the 10 mg/kg Q3W×2, followed by 6 mg/kg Q3W dosing regimen will likely provide the best risk/benefit ratio.

What is claimed is:

1. A method of administering an anti-CTLA-4 antibody, comprising administering one or more doses of the anti-CTLA-4 antibody to a subject, wherein each dose is independently about 0.1 mg/kg, 0.3 mg/kg, 1 mg/kg, 3 mg/kg, 6 mg/kg, 10 mg/kg, 15 mg/kg, or 20 mg/kg.

2. The method of claim 1, wherein the anti-CTLA-4 antibody is administered once about every 1, 2, 3, 4, 5, or 6 weeks.

3. The method of claim 2, wherein the anti-CTLA-4 antibody is administered once about every 3 weeks.

4. The method of any one of claims 1-3, wherein each dose of the anti-CTLA-4 antibody is about 6 mg/kg.

5. The method of any one of claims 1-3, wherein each dose of the anti-CTLA-4 antibody is about 10 mg/kg.

6. The method of any one of claims 1-3, comprising administering a first dose of about 10 mg/kg, a second dose of about 10 mg/kg, and one or more subsequent doses of about 1-6 mg/kg.

7. The method of claim 6, wherein each subsequent dose is about 6 mg/kg.

8. The method of claim 6, wherein each subsequent dose is about 3 mg/kg.

9. The method of any one of claims 1-3, wherein the anti-CTLA-4 antibody is administered to maintain a peak concentration (Cₘₐₓ) of about 200-300 μg/mL.

10. The method of claim 9, wherein the maintained Cₘₐₓ is about 225-250 μg/mL.

11. The method of 9 or 10, wherein a dose of the anti-CTLA-4 antibody administered to the subject is reduced as compared to an immediately preceding dose if (a) the Cₘₐₓ concentration of the anti-CTLA-4 antibody in a blood sample from the subject exceeds 300 μg/mL; (b) the subject experiences a limiting toxicity; or, (c) the subject is being treated for a cancer and achieves partial or complete response to treatment with the anti-CTLA-4 antibody according to Response Evaluation Criteria in Solid Tumors (RECIST) 1.1 criteria.

12. The method of any one of claims 1-11, wherein the anti-CTLA-4 antibody is administered intravenously.

13. The method of any one of claims 1-12, wherein the anti-CTLA-4 antibody comprises:
(a) a light chain variable region comprising a complementarity determining region (CDR) 1 comprising the amino acid sequence set forth in SEQ ID NO: 1; a CDR2 comprising the amino acid sequence set forth in any one of SEQ ID NOs: 2-4; and, a CDR3 comprising the amino acid sequence set forth in SEQ ID NO: 5; and,
(b) a heavy chain variable region comprising comprising a CDR1 comprising the amino acid sequence set forth in SEQ ID NO: 6; a CDR2 comprising the amino acid sequence set forth in any one of SEQ ID NOs: 7-9; and, a CDR3 comprising the amino acid sequence set forth in SEQ ID NO: 10.

14. The method of claim 13, wherein the anti-CTLA-4 antibody comprises a light chain variable region comprising a CDR2 comprising the sequence set forth in SEQ ID NO: 3 and heavy chain variable region comprising a CDR2 comprising the sequence set forth in SEQ ID NO: 9.

15. The method of claim 14, wherein the anti-CTLA-4 antibody comprises a light chain variable region comprising the sequence set forth in SEQ ID NO: 12 and a heavy chain variable region comprising the sequence set forth in SEQ ID NO: 16.

16. The method of claim 15, wherein the anti-CTLA-4 antibody comprises a light chain comprising the sequence set forth in SEQ ID NO: 23, and a heavy chain comprising the sequence set forth in SEQ ID NO: 21.

17. The method of any one of claims 1-16, wherein the subject has a cancer.

18. The method of claim 17, wherein the cancer is a solid tumor.

19. The method of claim 18, wherein the cancer is advanced or metatstatic.

20. The method of claim any one of claims 17-19, wherein the subject has previously exhibited failure or intolerance to standard of care for the cancer.

21. The method of claim 20, wherein the cancer is refractory or resistant to anti-PD-1/PD-L1 treatment.

22. The method of any one of claims 1-3, wherein the cancer is selected from the group consisting of melanoma, metastatic melanoma, PD (L)-1-refractory melanoma, non-small cell lung adenocarcinoma, metastatic NSCLC, NSCLC with driver mutations (for example, EGFR/ALK mutations or other targetable mutations), PD-1-refractory NSCLC, head and neck cancer, adenoid cystic carcinoma (which may be R/M), squamous carcinoma, triple negative (basal-type) breast cancer, pancreatic cancer, renal cell carcinoma, cervical cancer, endometrial cancer, colon cancer, hepatocellular carcinoma, other solid tumors, and metastatic colorectal cancer (which may have microsatellite instability).

23. A method of treating a cancer in a subject in need thereof, comprising administering one or more doses of an anti-CTLA-4 antibody to the subject, wherein each dose is independently about 0.1 mg/kg, 0.3 mg/kg, 1 mg/kg, 3 mg/kg, 6 mg/kg, 10 mg/kg, 15 mg/kg, or 20 mg/kg.

24. The method of claim 23, wherein the anti-CTLA-4 antibody is administered once about every 1, 2, 3, 4, 5, or 6 weeks.

25. The method of claim 24, wherein the anti-CTLA-4 antibody is administered once about every 3 weeks.

26. The method of any one of claims 23-25, wherein each dose of the anti-CTLA-4 antibody is about 6 mg/kg.

27. The method of any one of claims 23-25, wherein each dose of the anti-CTLA-4 antibody is about 10 mg/kg.

28. The method of any one of claims 23-25, comprising administering a first dose of about 10 mg/kg, a second dose of about 10 mg/kg, and one or more subsequent doses of about 1-6 mg/kg.

29. The method of claim 28, wherein each subsequent dose is about 6 mg/kg.

30. The method of claim 28, wherein each subsequent dose is about 3 mg/kg.

31. The method of any one of claims 23-25, wherein the anti-CTLA-4 antibody is administered to maintain a peak concentration (Cₘₐₓ) of about 200-300 μg/mL.

32. The method of claim 31, wherein the maintained Cₘₐₓ is about 225-300 μg/mL.

33. The method of claim 31 or 32, wherein a dose of the anti-CTLA-4 antibody administered to the subject is reduced as compared to an immediately preceding dose if (a) the Cₘₐₓ concentration of the anti-CTLA-4 antibody in a blood sample from the subject exceeds 300 μg/mL; (b) the subject experiences a limiting toxicity; or, (c) the subject achieves partial or complete response to treatment with the anti-CTLA-4 antibody according to Response Evaluation Criteria in Solid Tumors (RECIST) 1.1 criteria.

34. The method of any one of claims 23-33, wherein the anti-CTLA-4 antibody is administered intravenously.

35. The method of any one of claims 23-34, wherein the anti-CTLA-4 antibody comprises:
(a) a light chain variable region comprising a complementarity determining region (CDR) 1 comprising the amino acid sequence set forth in SEQ ID NO: 1; a CDR2 comprising the amino acid sequence set forth in any one of SEQ ID NOs: 2-4; and, a CDR3 comprising the amino acid sequence set forth in SEQ ID NO: 5; and,
(b) a heavy chain variable region comprising comprising a CDR1 comprising the amino acid sequence set forth in SEQ ID NO: 6; a CDR2 comprising the amino acid sequence set forth in any one of SEQ ID NOs: 7-9; and, a CDR3 comprising the amino acid sequence set forth in SEQ ID NO: 10.

36. The method of claim 35, wherein the anti-CTLA-4 antibody comprises a light chain variable region comprising a CDR2 comprising the sequence set forth in SEQ ID NO: 3 and heavy chain variable region comprising a CDR2 comprising the sequence set forth in SEQ ID NO: 9.

37. The method of claim 36, wherein the anti-CTLA-4 antibody comprises a light chain variable region comprising the sequence set forth in SEQ ID NO: 12 and a heavy chain variable region comprising the sequence set forth in SEQ ID NO: 16.

38. The method of claim 37, wherein the anti-CTLA-4 antibody comprises a light chain comprising the sequence set forth in SEQ ID NO: 23, and a heavy chain comprising the sequence set forth in SEQ ID NO: 21.

39. The method of any one of claims 23-38, wherein the cancer is a solid tumor.

40. The method of claim 39, wherein the cancer is advanced or metatstatic.

41. The method of claim any one of claims 23-40, wherein the subject has previously exhibited failure or intolerance to standard of care for the cancer.

42. The method of claim 41, wherein the cancer is refractory or resistant to anti-PD-1/PD-L1 treatment.

43. The method of any one of claims 23-25, wherein the cancer is selected from the group consisting of: melanoma, metastatic melanoma, PD (L)-1-refractory melanoma, non-small cell lung adenocarcinoma, metastatic NSCLC, NSCLC with driver mutations (for example, EGFR/ALK mutations or other targetable mutations), PD-1-refractory NSCLC, head and neck cancer, adenoid cystic carcinoma (which may be R/M), squamous carcinoma, triple negative (basal-type) breast cancer, pancreatic cancer, renal cell carcinoma, cervical cancer, endometrial cancer, colon cancer, hepatocellular carcinoma, other solid tumors, and metastatic colorectal cancer (which may have microsatellite instability).


SEQUENCE LISTING (24 sequences)

SEQ ID NO: 1 - protein, 11 aa, synthetic construct
RASENIYSNLA

SEQ ID NO: 2 - protein, 7 aa, synthetic construct
AATNLQS

SEQ ID NO: 3 - protein, 7 aa, synthetic construct
AATNLQD

SEQ ID NO: 4 - protein, 7 aa, synthetic construct
AATSLQS

SEQ ID NO: 5 - protein, 9 aa, synthetic construct
QHLWGTPYT

SEQ ID NO: 6 - protein, 10 aa, synthetic construct
GFSLTSYGLS

SEQ ID NO: 7 - protein, 17 aa, synthetic construct
YIWYDGNTNFHPSLKSR

SEQ ID NO: 8 - protein, 17 aa, synthetic construct
YIWYDGNTNFHSSLKSR

SEQ ID NO: 9 - protein, 17 aa, synthetic construct
YIWYDGNTNFHSPLKSR

SEQ ID NO: 10 - protein, 17 aa, synthetic construct
TEGHYYGSNYGYYALDY

SEQ ID NO: 11 - protein, 107 aa, synthetic construct
DIQMTQSPSSLSASVGDRVTITCRASENIYSNLAWYQQKPGKAPKLLLYAATNLQSGVPSRFSGSGSGTDFTLTISSLQPEDFATYYCQHLWGTPYTFGGGTKLEIK

SEQ ID NO: 12 - protein, 107 aa, synthetic construct
DIQMTQSPSSLSASVGDRVTITCRASENIYSNLAWYQQKQGKAPKLLLYAATNLQDGVPSRFSGSGSGTDYTLTISSLQPEDFATYFCQHLWGTPYTFGQGTKLEIK

SEQ ID NO: 13 - protein, 107 aa, synthetic construct
DIQMTQSPSSLSASVGDRVTITCRASENIYSNLAWYQQKPGKAPKLLIYAATSLQSGVPSRFSGSGSGTDFTLTISSLQPEDFATYYCQHLWGTPYTFGGGTKVEIK

SEQ ID NO: 14 - protein, 125 aa, synthetic construct
QVQLQESGPGLVKPSETLSLTCTVSGFSLTSYGLSWIRQPPGKGLEWIGYIWYDGNTNFHPSLKSRVTISKDTSKNQFSLKLSSVTAADTAVYYCAKTEGHYYGSNYGYYALDYWGQGTSVTVSS

SEQ ID NO: 15 - protein, 125 aa, synthetic construct
QVQLQESGPGLVKPSETLSLTCTVSGFSLTSYGLSWIRQPPGKGLEWIGYIWYDGNTNFHSSLKSRVTISKDTSKSQVSLKLSSVTAADTAVYYCAKTEGHYYGSNYGYYALDYWGQGTLVTVSS

SEQ ID NO: 16 - protein, 125 aa, synthetic construct
QVQLQESGPGLVKPSETLSLTCTVSGFSLTSYGLSWIRQPPGKGLEWIGYIWYDGNTNFHSPLKSRVTISVDTSKNQFSLKLSSVTAADTAVYYCAKTEGHYYGSNYGYYALDYWGQGTLVTVSS

SEQ ID NO: 17 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 454 aa, synthetic construct
QVQLQESGPGLVKPSETLSLTCTVSGFSLTSYGLSWIRQPPGKGLEWIGYIWYDGNTNFHPSLKSRVTISKDTSKNQFSLKLSSVTAADTAVYYCAKTEGHYYGSNYGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 454 aa, synthetic construct
QVQLQESGPGLVKPSETLSLTCTVSGFSLTSYGLSWIRQPPGKGLEWIGYIWYDGNTNFHSSLKSRVTISKDTSKSQVSLKLSSVTAADTAVYYCAKTEGHYYGSNYGYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 454 aa, synthetic construct
QVQLQESGPGLVKPSETLSLTCTVSGFSLTSYGLSWIRQPPGKGLEWIGYIWYDGNTNFHSPLKSRVTISVDTSKNQFSLKLSSVTAADTAVYYCAKTEGHYYGSNYGYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 214 aa, synthetic construct
DIQMTQSPSSLSASVGDRVTITCRASENIYSNLAWYQQKPGKAPKLLLYAATNLQSGVPSRFSGSGSGTDFTLTISSLQPEDFATYYCQHLWGTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 214 aa, unidentified
DIQMTQSPSSLSASVGDRVTITCRASENIYSNLAWYQQKQGKAPKLLLYAATNLQDGVPSRFSGSGSGTDYTLTISSLQPEDFATYFCQHLWGTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 214 aa, synthetic construct
DIQMTQSPSSLSASVGDRVTITCRASENIYSNLAWYQQKPGKAPKLLIYAATSLQSGVPSRFSGSGSGTDFTLTISSLQPEDFATYYCQHLWGTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC